(19) United States
(12) Patent Application Publication
(10) Pub. No.: US 2024/0309389 A1
(43) Pub. Date: Sep. 19, 2024

(54) Chimeric FCeRI a-Chain Gene, Chimeric FCeRI a-Chain Protein, Cells, Analysis Kit and Analysis Method

(71) Applicant: TEIKYO HEISEI UNIVERSITY, Tokyo (JP)

(72) Inventors: Ryosuke Nakamura, Kawasaki-shi (JP); Haruyo Akiyama, Tokyo (JP); Satoshi Tadokoro, Chigasaki-shi (JP); Kenichi Kumasaka, Chigasaki-shi (JP)

(21) Appl. No.: 18/279,709

(22) PCT Filed: Feb. 24, 2022
(86) PCT No.: PCT/JP2022/007612

§ 371 Date: Aug. 31, 2023

(30) Foreign Application Priority Data
Mar. 1, 2021 (JP) 2021 031757

(51) Int. Cl. C12N 15/62; C07K 14/735; C12N 15/63; C12Q 1/02

(52) U.S. Cl. CPC C12N 15/62; C07K 14/70535; C12N 15/63; C12Q 1/02; C07K 2319/03

(57) ABSTRACT

Provided herein is a chimeric FcεRI α-chain gene including a first base sequence encoding an extracellular domain of a human FcεRI α-chain and a second base sequence encoding at least a transmembrane domain of a non-human animal FcεRI α-chain. Also provided herein is a chimeric FcεRI α-chain protein that is an expression product of the chimeric FcεRI α-chain gene, and a non-human animal cell including the chimeric FcεRI α-chain gene. Also provided herein is an analysis kit including the cells and an analysis method using the cells.

### CROSS REFERENCE TO RELATED APPLICATIONS

This application is the United States national phase of International Patent Application No. PCT/JP2022/007612 filed Feb. 24, 2022, and claims priority to Japanese Patent Application No. 2021-031757 filed Mar. 1, 2021, the disclosures of which are hereby incorporated by reference in their entireties.

The Sequence Listing associated with this application is filed in electronic format via Patent Center and is hereby incorporated by reference into the specification in its entirety. The name of the computer-readable form (CRF) containing the Sequence Listing is 2305807_ST25.txt. The size of the file is 13,414 bytes, and the file was created on Aug. 21, 2023.

### BACKGROUND OF THE INVENTION

### Technical Field

The present invention relates to chimeric FcεRI α-chain genes, chimeric FcεRI α-chain proteins, cells, analysis kits, and analysis methods. More particularly, the present invention relates to chimeric FcεRI α-chain genes, chimeric FcεRI α-chain proteins, cells, and kits used to perform allergy-related tests, and analysis methods related to allergy.

### Background Art

IgE, which is involved in allergic reactions, is an antibody that exists in very small amounts in serum. IgE binds very strongly to the α-chain of high-affinity IgE receptors (herein, the high-affinity IgE receptor is also referred to as “FcεRI”) expressed by cells such as mast cells and basophils (Ka=10¹⁰M⁻¹). FcεRI may comprise one α-chain, one β-chain, and a homodimer of γ-chains. In many animal species, these subunits are expressed on the cell membrane surface only after forming a tetramer, but in humans, an expression mode that does not involve a β-chain is also known.

When B cells produce allergen (antigen)-specific IgE, the IgE enters the bloodstream and binds to FcεRI expressed by peripheral immune cells, particularly mast cells and basophils, to sensitize these cells. When these IgE-sensitized cells are exposed to a specific antigen, the antigen and IgE bind. At this time, if multiple IgE epitopes are present in one antigen molecule, multiple IgE antibodies will bind via the antigen, and multiple FcεRIs will be “crosslinked” accordingly.

Cross-linking of FcεRI triggers, through the activation of enzymes such as tyrosine kinases and adapter molecules that associate with the intracellular domain of the receptor, cellular responses such as calcium ion influx, degranulation of chemical mediators such as histamine, and increased production of lipid chemical mediators, resulting in an immediate allergic response. In addition, through activation of certain transcription factors, gene expression of, for example, cytokines and chemokines, is induced, leading to a later inflammatory response.

Several testing methods for allergy have been proposed so far. For example, Patent Literature 1 below discloses a method in which cells having an Fc receptor with affinity for human IgE on the cell membrane and having a promoter and a reporter gene in this order under the control of an enhancer capable of binding to a transcription factor are incubated in the presence of a biological sample from a test subject; a test substance and the cells after the incubation are brought into contact; and an increase in the expression of the reporter gene in the cells contacted with the test substance is confirmed; thereby examining whether the test substance is an allergen for the test subject.

### CITATION LIST

### Patent Literature

Patent Literature 1: JP 2011-19467 A

### SUMMARY OF THE INVENTION

### Technical Problem

There is a demand for further improvement in allergy test sensitivity. If the sensitivity can be improved, it will be useful for various tests relating to allergy, such as evaluation based on the biological significance of patient IgE, detection of trace allergens, and high-throughput screening of anti-allergic substances. Accordingly, the main object of the present invention is to increase the sensitivity of allergy tests, particularly to increase the sensitivity of IgE detection techniques.

### Solution to Problem

The present invention provides a chimeric FcεRI α-chain gene comprising a first base sequence that may encode an extracellular domain of a human FcεRI α-chain and a second base sequence that may encode at least a trans-cellularmembrane domain of a non-human animal FcεRI α-chain.

The first base sequence may comprise a base sequence having 70% or more sequence identity with the base sequence of SEQ ID NO: 7.

The second base sequence may comprise a base sequence having 70% or more sequence identity with the base sequence of SEQ ID NO: 5.

The second base sequence may further comprise a base sequence having 70% or more sequence identity with the base sequence of SEQ ID NO: 6.

The chimeric FcεRI α-chain gene may further comprise a third base sequence encoding a signal sequence for expressing, on the cell membrane, the chimeric FcεRI α-chain produced based on the gene.

The third base sequence may include a base sequence having 70% or more sequence identity with the base sequence of SEQ ID NO: 2.

The chimeric FcεRI α-chain gene may have a base sequence having 50% or more sequence identity with the base sequence of SEQ ID NO: 1.

The non-human animal may be a rodent animal.

The present invention also provides a chimeric FcεRI α-chain protein comprising an extracellular domain of a human FcεRI α-chain and at least a transmembrane domain of a non-human animal FcεRI α-chain.

The extracellular domain may comprise an amino acid sequence having 70% or more sequence identity with the amino acid sequence of SEQ ID NO: 14.

The transmembrane domain may comprise an amino acid sequence having 70% or more sequence identity with the amino acid sequence of SEQ ID NO: 12.

The chimeric FcεRI α-chain protein further may comprise an intracellular domain of a non-human animal FcεRI α-chain; and
the intracellular domain may comprise an amino acid sequence having 70% or more sequence identity with the amino acid sequence of SEQ ID NO: 13.

The chimeric FcεRI α-chain protein may have an amino acid sequence that has 70% or more sequence identity with the amino acid sequence of SEQ ID NO: 8.

The present invention also provides a vector comprising the chimeric FcεRI α-chain gene.

The present invention also provides a cell of the non-human animal comprising the chimeric FcεRI α-chain gene, the chimeric FcεRI α-chain protein, or the vector.

The cell may further comprise the FcεRI γ-chain gene of the non-human animal cell.

The cell may form a complex comprising the chimeric FcεRI α-chain produced based on the chimeric FcεRI α-chain gene and the FcεRI γ-chain of the cell.

The cell may further comprise a reporter gene whose expression is induced by cross-linking between the complex.

The present invention also provides an analysis kit comprising the cells.

The present invention also provides an analysis method using the cells.

The analysis method may comprise performing an incubation step of incubating the cells and an analysis step of analyzing the response of the cells.

The incubation step may comprise incubating the cells in an incubation material.

The analysis step may comprise performing at least one selected from the group consisting of evaluation of allergenicity, evaluation of the presence or absence or risk of allergy, analysis of IgE, evaluation of allergy suppressive activity, and drug screening, based on the response.

The analysis step may comprise evaluating the presence or absence or risk of type I allergy.

### BRIEF DESCRIPTION OF THE DRAWINGS

FIG. 1 is a schematic diagram for explaining the cells of the present invention.

FIG. 2 is a schematic diagram for explaining the chimeric FcεRI α-chain protein of the present invention.

FIG. 3 is a diagram showing a configuration example of the chimeric FcεRI α-chain gene and chimeric FcεRI α-chain protein of the present invention.

FIG. 4 is a diagram showing the base sequence of the chimeric FcεRI α-chain gene (SEQ ID NO: 1) used in Examples and the amino acid sequence (SEQ ID NO: 8) encoded by the gene.

FIG. 5 is the base sequence of SEQ ID NO: 15.

FIG. 6 is a diagram illustrating sequences for gene insertion; the figure shows excerpts (nucleotides 1-16 and 747-757) of SEQ ID NO: 16.

FIG. 7 is a diagram showing the measurement results of luminescence intensity.

FIG. 8 is a diagram showing the measurement results by a flow cytometer.

FIG. 9 is a diagram showing the measurement results of luminescence intensity.

FIG. 10 is a diagram showing the measurement results of luminescence intensity.

FIG. 11 is a diagram showing the measurement results of absorbance.

FIG. 12 is a diagram showing the measurement results of luminescence intensity.

FIG. 13 is a diagram showing the measurement results of luminescence intensity.

FIG. 14 is a diagram showing the measurement results of luminescence intensity.

FIG. 15 is a diagram showing the measurement results of luminescence intensity.

FIG. 16 is a diagram showing the measurement results of luminescence intensity.

FIG. 17 is a diagram showing the measurement results of luminescence intensity.

FIG. 18 is a diagram showing the measurement results of luminescence intensity.

FIG. 19 is a schematic diagram for explaining the structure and function of the chimeric FcεRI α-chain gene and the chimeric FcεRI α-chain protein expressed from the gene used in Examples.

FIG. 20 is a diagram showing the measurement results of luminescence intensity.

FIG. 21 is a diagram showing the measurement results of luminescence intensity.

FIG. 22 is a diagram showing the measurement results of luminescence intensity.

FIG. 23 is a diagram showing the measurement results of luminescence intensity.

### DESCRIPTION OF THE INVENTION

Preferred embodiments for carrying out the present invention will be described below.

The embodiments described below are representative embodiments of the present invention, and the scope of the present invention is not limited only to these embodiments.

The present invention will be described in the following order.
1. Description of the present invention
2. First embodiment (chimeric FcεRI α-chain gene)
   (1) First base sequence
   (2) Second base sequence
   (3) Third base sequence
   (4) Entire chimeric FcεRI α-chain gene
3. Second embodiment (chimeric FcεRI α-chain protein)
   (1) Extracellular domain
   (2) Transmembrane domain and intracellular domain
   (3) Signal sequence
   (4) Chimeric FcεRI α-chain protein
4. Third embodiment (vector)
5. Fourth embodiment (cell)
6. Fifth embodiment (analysis kit)
7. Sixth embodiment (analysis method)
8. Examples
   (1) Synthesis of chimeric FcεRI α-chain gene
   (2) Introduction of chimeric FcεRI α-chain gene into RBL-NL4 cells
   (3) Luciferase expression in cells into which chimeric FcεRI α-chain gene was introduced
   (4) Confirmation of IgE-binding ability to cells into which chimeric FcεRI α-chain gene was introduced
   (5) Examination of test conditions suitable for the cells of the present invention (6) Test using allergy patient serum (peanut allergy)
   (7) Comparison between the cells of the present invention and other cells (allergy test, egg allergy)
   (8) Comparison between the cells of the present invention and other cells (Evaluation of effectiveness of allergic reaction inhibitors)
   (9) Stability and reproducibility precision

### 1. Description of the Present Invention

Allergy test methods generally used in clinical practice at present can be divided into, for example, in vivo test methods, ex vivo test methods, and in vitro test methods, each of which has advantages and disadvantages. Since the in vivo test method administers the allergen to the patient himself/herself, it has the highest reliability, but there is a problem that it imposes a burden on the patient. In ex vivo test methods, a patient's basophils can be removed from the body and an allergen may be administered to the basophils. The ex vivo test method is highly reliable because it uses basophil activation (for example, histamine release or CD203c/CD63 expression on the cell surface) as an index. However, since the ex vivo test method uses whole blood, there is a problem that the sample cannot be stored. One of the most frequently used in vitro test methods is the ImmunoCAP method. The ImmunoCAP method can be performed using the patient's serum or plasma. The ImmunoCAP method is simple and relatively sensitive, but has the problem of a large number of false positives. This problem is thought to occur when there is only one IgE epitope on the allergen. This problem is also thought to be due to the low binding affinity between allergens and IgE and the inability to maintain cross-linking of FcεRI.

While the ex vivo test method described above quantifies patient basophil activation, this method can be modified slightly to examine IgE-dependent basophil activation in patient sera. That is, in the modified method, first, peripheral blood basophils are isolated from a healthy person, and the isolated basophils are treated with acid to dissociate the bound IgE from the basophils. After that, the basophils are passively sensitized with subject-derived serum IgE, and antigen-specific activation of basophils by the sensitization is examined. However, this method is troublesome, and since peripheral blood from the same healthy person cannot always be used, there is a problem that the test lacks reproducibility.

As a method to solve such problems, for example, a method in which a cultured cell line in which human FcεRI is stably expressed in RBL-2H3 cells known as a rat cultured mast cell line is used, and this is sensitized with allergy patient serum, and the amount of degranulation when a specific antigen is added is measured has been devised by several groups. This method was epoch-making in that it was possible to sensitize cultured mast cells expressing human FcεRI with IgE in patient serum and detect cross-linking of FcεRI by antigen. However, this method may suffer from low sensitivity of degranulation measurement and high probability of false negatives.

These problems can be addressed by the method described in Patent Literature 1 above. The method described in Patent Literature 1 uses cells that have an Fc receptor that has an affinity for human IgE on the cell membrane, and that have a promoter and a reporter gene in this order under the control of an enhancer that can be bound by a transcription factor. As an example of such cells, Patent Literature 1 above discloses a cell line RS-ATL8 cells in which a reporter gene that induces the expression of a firefly luciferase gene in a transcription factor Nuclear Factor of Activated T-cells (NF-AT)-dependent manner was introduced into RBL-SX38 cells, which are cultured cell lines in which human FcεRI is stably expressed in RBL-2H3 cells. In the method described in Patent Literature 1, the cells are sensitized with IgE by, for example, overnight culture with 100-fold diluted patient serum or the like, and after the sensitization, the cells are brought into contact with a specific antigen. Such contact induces cross-linking of FcεRI. The cross-linking results in expression of luciferase via intracellular signaling involving the transcription factor. This allows very sensitive detection of antigens or specific IgE. This method is also called “IgE-Crosslinking-induced Luciferase Expression (ExiLE) method”. The method has a very simple protocol and is suitable for screening a large number of specimens. Furthermore, the method has very excellent performance, for example, the area under the curve exceeds 0.97 in ROC curve analysis for patients with egg white allergy. Researchers around the world are already using this method, and as of August 2019, it is being used in 16 countries, mainly in Europe and the united States.

Although the method described in Patent Literature 1 can detect antigens and specific IgE with high sensitivity, there is a demand for further improvement in sensitivity. Further sensitivity improvements would be useful, for example, for biological significance-based evaluation of patient IgE, detection of trace allergens, and high-throughput screening of anti-allergens.

The present inventors have found that a chimeric FcεRI α-chain gene comprising a first base sequence encoding an extracellular domain of a human FcεRI α-chain and a second base sequence encoding at least a transmembrane domain of a non-human animal FcεRI α-chain is extremely useful for improving the sensitivity of an allergy test. For example, the non-human animal cells (e.g., mast cells) into which the chimeric FcεRI α-chain gene has been introduced are suitable for use in tests relating to human allergy, and can be used, for example, as a material for testing human allergy with extremely high sensitivity. For example, the chimeric FcεRI α-chain gene may be used, for example, in the method described in Patent Literature 1 above, and particularly introduced into cells used in the method described in Patent Literature 1 above.

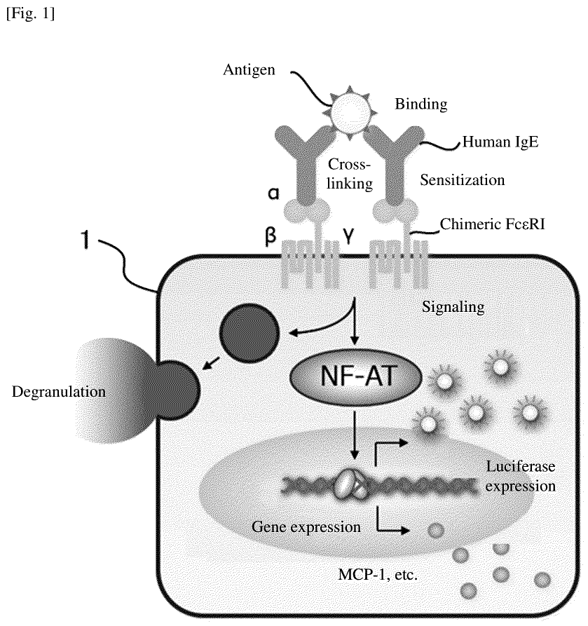

Examples of cells having the chimeric FcεRI α-chain gene of the present invention and the mechanism of antigen or IgE detection by such cells are described below with reference to FIGS. 1 and 2.

Cell 1 shown in FIG. 1 is a non-human animal cell. Cell 1 contains the chimeric FcεRI α-chain gene of the present invention and a reporter gene. Cell 1 further comprises the FcεRI γ-chain gene of the non-human animal cell. The non-human animal cell may further comprise the FcεRI β-chain gene of the non-human animal cell.

The chimeric FcεRI α-chain gene, the FcεRI β-chain gene and the FcεRI γ-chain gene are expressed in cell 1 to produce chimeric FcεRI α-chain protein, FcεRI β-chain protein and FcεRI γ-chain protein. Chimeric FcεRI, which is a tetramer formed by association of one chimeric FcεRI α-chain protein, one FcεRI β-chain protein, and two FcεRI γ-chain proteins (homodimer), is expressed on the cell membrane surface of cell 1, as shown in FIG. 1. In dendritic cells, chimeric FcεRI is expressed in a trimer conformation consisting of one α-chain and two γ-chains.

A more detailed schematic of the chimeric FcεRI is shown in FIG. 2A. As shown in FIG. 2A, the chimeric FcεRI is a tetramer consisting of a chimeric FcεRI α-chain protein (indicated as ₕ₊ᵣα in the figure), an FcεRI β-chain protein (ᵣβ in the figure), and a homodimer of the FcεRI γ-chain protein (ᵣγ in the figure). The α-chain protein comprises a first portion derived from the human FcεRI α-chain (the portion indicated by dots in the figure) and a second portion derived from the non-human animal FcεRI α-chain (the portion indicated by gray in the figure).

It should be understood that FIGS. 1 and 2 are schematic diagrams for explanation and do not reflect actual conditions.

The first portion comprises the extracellular domain of the human FcεRI α-chain. The extracellular domain may literally be exposed outside the cell. The extracellular domain comprises the IgE binding region of the human FcεRI α-chain.

For example, as shown in FIG. 2A, the extracellular domain exists extracellularly, and the α-chain protein can bind to human IgE (ₕIgE) via the IgE-binding region contained in the extracellular domain.

The second portion comprises at least the transmembrane domain of the non-human animal FcεRI α-chain. In addition, the transmembrane domain comprises one or more amino acid residues that contribute to binding to the non-human animal FcεRI γ-chain, e.g., exert such binding properties. The second portion may further comprise the intracellular domain of the non-human animal FcεRI α-chain.

For example, as shown in FIG. 2A, the α-chain protein can bind to the γ-chain protein via the one or more amino acid residues of the transmembrane domain. In addition, as shown in the figure, the α-chain protein stays in the cell membrane due to the transmembrane domain.

The FcεRI β-chain protein may be an expression product of the FcεRI β-chain gene endogenously present in the non-human animal.

The FcεRI γ-chain protein may also be an expression product of the FcεRI γ-chain gene endogenously present in the non-human animal.

A schematic diagram of FcεRI expressed by RS-ATL8 cells described above is shown in FIG. 2B. RS-ATL8 cells are cells obtained by stably introducing an NF-AT-dependent firefly luciferase expression vector into RBL-SX38 cells, in which human FcεRI is stably expressed in RBL-2H3 cells. As shown in FIG. 2B, the α- and γ-chains of FcεRI expressed in RS-ATL8 cells are of human FcεRI (dotted parts), and the β-chain appears to be of rat FcεRI (grey-colored parts). FcεRI expressed in RS-ATL8 cells can bind human IgE since its α-chain is that of human FcεRI.

The α-chain of FcεRI in RS-ATL8 cells is wholly human FcεRI, whereas the chimeric FcεRI α-chain protein of the present invention in FIG. 2A has the extracellular domain of human FcεRI and the transmembrane domain of non-human animal FcεRI.

A schematic diagram of FcεRI expressed by the RBL-2H3 cells described above is also shown in FIG. 2C. The α-chain, β-chain, and γ-chain of the FcεRI are all derived from rat, and the α-chain binds to rat IgE.

As shown in FIG. 1, type I allergy is caused by (1) binding of FcεRI and IgE (sensitization), (2) binding of antigen (allergen) against IgE and IgE bound to FcεRI (binding), (3) cross-linking between multiple IgE-binding FcεRIs bound to one antigen (cross-linking), (4) release of physiologically active substances such as histamine and serotonin from cells (degranulation), and (5) systemic response due to physiologically active substances.

By confirming the gene expression of the reporter gene whose expression is induced by cross-linking between multiple IgE-binding FcεRIs in (3) above, for example, an antigen (especially a specific antigen) that binds to IgE can be detected.

### 2. First Embodiment (Chimeric FcεRI α-Chain Gene)

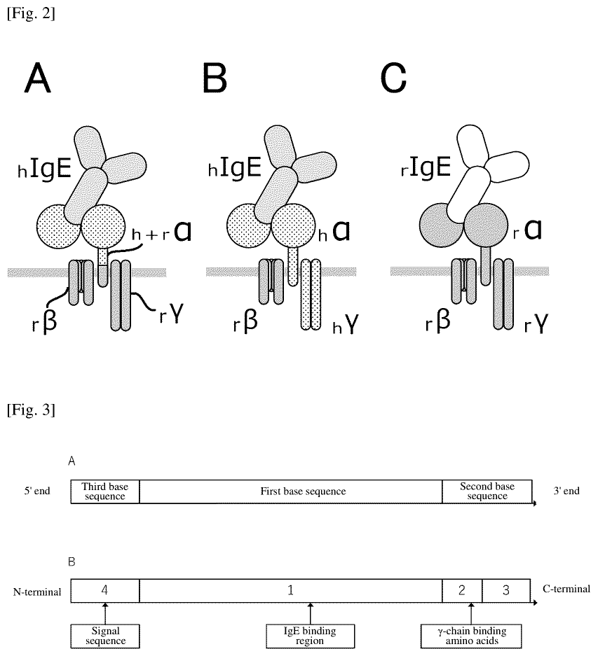

An example construction of a chimeric FcεRI α-chain gene of the present invention is described below with reference to FIG. 3A. FIG. 3A is a schematic diagram for explaining the construction of the chimeric FcεRI α-chain gene of the present invention. In FIG. 3A, the left side of the page is the 5′ end of the gene, and the right side of the page is the 3′ end of the gene.

In addition, FIG. 3B is shown below the schematic diagram, and FIG. 3B shows a configuration example of the chimeric FcεRI α-chain protein of the present invention produced by expression of the chimeric FcεRI α-chain gene of the present invention. In FIG. 3B, the left side of the page is the N-terminal of the protein, and the right side of the page is the C-terminal of the protein.

As shown in FIG. 3A, the chimeric FcεRI α-chain gene of the present invention comprises a first base sequence encoding the extracellular domain of the human FcεRI α-chain and a second base sequence encoding at least the transmembrane domain of the non-human animal FcεRI α-chain. The second base sequence particularly encodes a transmembrane domain and an intracellular domain of the non-human animal FcεRI α-chain, and the transmembrane domain may exert a binding property to the non-human animal FcεRI γ-chain.

The chimeric FcεRI α-chain gene of the present invention may further comprise a third base sequence encoding a signal sequence for expressing the chimeric FcεRI α-chain produced based on the gene on the cell membrane.

A chimeric FcεRI α-chain protein produced by expression of the chimeric FcεRI α-chain gene of the present invention may comprise extracellular domain 1 of the human FcεRI α-chain and transmembrane domain 2 and intracellular domain 3 of the non-human animal FcεRI α-chain, as shown in FIG. 3B. The extracellular domain 1 may comprise an IgE binding region. The transmembrane domain 2 can exhibit binding properties to the non-human animal FcεRI γ-chain and can comprise one or more amino acid residues that exhibit such binding properties. The chimeric FcεRI α-chain protein may further comprise a signal sequence 4 to allow the protein to express on the cell membrane.

As described above, the first base sequence contained in the chimeric FcεRI α-chain gene corresponds to the extracellular domain contained in the chimeric FcεRI α-chain protein. The second base sequence corresponds to the transmembrane domain and the intracellular domain. The third base sequence corresponds to the signal sequence.

The first to third base sequences may be arranged in the order of the third base sequence, the first base sequence, and the second base sequence from the 5′ end, as shown in FIG. 3A.

The extracellular domain, the transmembrane domain, the intracellular domain, and the signal sequence may be arranged in the order of the signal sequence, the extracellular domain, the transmembrane domain, and the intracellular domain from the N-terminal, as shown in FIG. 3B. Also, one or more amino acid residues may or may not be inserted between these regions.

### (1) First Base Sequence

The first base sequence encodes the extracellular domain of the human FcεRI α-chain, as described above. The extracellular domain comprises the IgE binding region of the human FcεRI α-chain. The IgE binding region may be a region that binds human IgE.

The first base sequence preferably has 70% or more, more preferably 80% or more, even more preferably 85% or more, particularly preferably 90% or more, 92% or more, 94% or more, 95% or more, 96% or more, 97% or more, 98% or more, or 99% or more sequence identity with the base sequence of SEQ ID NO: 7. The amino acid sequence encoded by the base sequence is preferable for binding to IgE and functions as an IgE-binding region.

SEQ ID NO: 7 is as follows. SEQ ID NO: 7 is a base sequence corresponding to amino acid residue numbers 110 to 183 in the amino acid sequence of SEQ ID NO: 8 (chimeric FcεRI α-chain protein of the present invention) described below.

SEQ ID NO: 7:
TCC GAT TGG CTG CTC CTG CAG GCC TCT
GCT GAG GTG GTC ATG GAA GGC CAG CCA
CTC TTC CTG AGG TGT CAC GGA TGG CGG
AAC TGG GAC GTG TAC AAG GTC ATC TAC
TAT AAA GAT GGG GAG GCC CTG AAG TAT
TGG TAC GAA AAC CAT AAC ATC AGT ATC
ACT AAC GCA ACC GTG GAG GAC TCA GGC
ACA TAC TAT TGT ACT GGA AAA GTC TGG
CAG CTC

In a more preferred embodiment, the first base sequence may have 70% or more, more preferably 80% or more, even more preferably 85% or more, particularly preferably 90% or more, 92% or more, 94% or more, 95% or more, 96% or more, 97% or more, 98% or more, or 99% or more sequence identity with the base sequence of SEQ ID NO: 3 below. The amino acid sequence encoded by the base sequence is preferable for binding to IgE, and is also preferable for maintaining the structure for providing IgE binding. The amino acid sequence encoded by the base sequence may constitute the entire extracellular domain of the chimeric FcεRI α-chain protein of the present invention.

SEQ ID NO: 3 is a base sequence corresponding to amino acid residue numbers 26 to 205 in the amino acid sequence of SEQ ID NO: 8 (chimeric FcεRI α-chain protein of the present invention) described below.

SEQ ID NO: 3:
GTC CCC CAG AAG CCC AAG GTG TCT CTC
AAC CCC CCT TGG AAT AGA ATC TTT AAA
GGG GAG AAC GTG ACC CTG ACA TGC AAT
GGT AAC AAT TTC TTT GAA GTC AGC TCC
ACT AAG TGG TTT CAC AAC GGG TCA CTC
AGC GAG GAA ACA AAT TCT AGT CTG AAC
ATT GTG AAT GCC AAG TTC GAG GAC AGC
GGT GAA TAC AAA TGC CAG CAT CAG CAG
GTG AAC GAG TCC GAA CCT GTG TAT CTG
GAG GTC TTT TCC GAT TGG CTG CTC CTG
CAG GCC TCT GCT GAG GTG GTC ATG GAA
GGC CAG CCA CTC TTC CTG AGG TGT CAC
GGA TGG CGG AAC TGG GAC GTG TAC AAG
GTC ATC TAC TAT AAA GAT GGG GAG GCC
CTG AAG TAT TGG TAC GAA AAC CAT AAC
ATC AGT ATC ACT AAC GCA ACC GTG GAG
GAC TCA GGC ACA TAC TAT TGT ACT GGA
AAA GTC TGG CAG CTC GAT TAC GAG AGC
GAA CCA CTG AAT ATC ACC GTG ATT AAG
GCT CCC AGG GAG AAA TAT TGG CTC CAG

The first base sequence may also encode two amino acid sequences that function as immunoglobulin domains.

One of the two amino acid sequences is the amino acid sequence of amino acid residue numbers 44 to 108 in the amino acid sequence of SEQ ID NO: 8 (chimeric FcεRI α-chain protein of the present invention). The base sequence encoding the amino acid sequence corresponds to nucleotide numbers 130-324 in SEQ ID NO: 1, for example.

The other of the two amino acid sequences is the amino acid sequence of amino acid residue numbers 121-194 in the amino acid sequence of SEQ ID NO: 8. The base sequence encoding the amino acid sequence corresponds to, for example, nucleotide numbers 361-582 in SEQ ID NO: 1.

A region between these two amino acid sequences that functions as an immunoglobulin domain is also called a linker region. The linker region is the amino acid sequence of amino acid residue numbers 109-120 in the amino acid sequence of SEQ ID NO: 8. The base sequence encoding the linker region corresponds to nucleotide numbers 325-360 in SEQ ID NO: 1, for example.

Sequence identity as described above for SEQ ID NO: 3 is also allowed for these base sequences.

As used herein, the term “sequence identity” of base sequences (or amino acid sequences) is a percentage obtained by aligning two base sequences (or amino acid sequences) to be compared so that the bases (or amino acid residues) of the two base sequences (or amino acid sequences) match as much as possible, and dividing the number of matched bases (or the number of matched amino acid residues) by the total number of bases (or the total number of amino acid residues). The alignment and calculation of the percentages may be performed using well-known algorithms such as BLAST.

### (2) Second Base Sequence

The second base sequence encodes at least the transmembrane domain of the non-human animal FcεRI α-chain, as described above. The transmembrane domain has a function of binding to the γ-chain. In order to exert its function, the transmembrane domain may comprise amino acid residues involved in binding to the γ-chain of non-human animal FcεRI. For example, Asp contained in the transmembrane domain described below may be involved in the binding.

The non-human animal is, for example, a rodent animal, preferably a rat, mouse or hamster, more preferably a rat or mouse, particularly preferably a rat.

In addition to the transmembrane domain, the second base sequence may further encode a region of the non-human animal FcεRI α-chain that comprises the intracellular domain.

Particularly preferably, the second base sequence may encode a region comprising both the transmembrane domain and the intracellular domain of the non-human animal FcεRI α-chain. The transmembrane domain may have a function of binding to the non-human animal FcεRI γ-chain, and may comprise the Asp for exhibiting the function. The Asp may be Asp at amino acid number 219 in SEQ ID NO: 12 described below.

The second base sequence preferably comprises a base sequence having 70% or more, more preferably 80% or more, even more preferably 85% or more, particularly preferably 90% or more, 92% or more, 94% or more, 95% or more, 96% or more, 97% or more, 98% or more, or 99% or more sequence identity with the base sequence of SEQ ID NO: 5, and a base sequence having 70% or more, more preferably 80% or more, even more preferably 85% or more, particularly preferably 90% or more, 92% or more, 94% or more, 95% or more, 96% or more, 97% or more, 98% or more, or 99% or more sequence identity with the base sequence of SEQ ID NO: 6. Amino acid sequences encoded by such base sequences are preferred for binding to non-human animal FcεRI γ-chains.

The base sequence having the sequence identity with the base sequence of SEQ ID NO: 5 corresponds to the transmembrane domain of the non-human animal FcεRI α-chain.

The base sequence having the sequence identity with the base sequence of SEQ ID NO: 6 corresponds to the intracellular domain of the non-human animal FcεRI α-chain.

SEQ ID NO: 5 and SEQ ID NO: 6 are as follows.

SEQ ID NO: 5 is a base sequence corresponding to amino acid residue numbers 206 to 224 (transmembrane domain) in the amino acid sequence of SEQ ID NO: 8 (chimeric FcεRI α-chain protein of the present invention) described below.

SEQ ID NO: 6 is a base sequence corresponding to amino acid residue numbers 225 to 246 (intracellular domain) in the amino acid sequence.

SEQ ID NO: 5:
CTG ATC TTC CCT TCT CTC GCA GTG ATT
CTG TTT GCC GTC GAC ACC GGA CTG TGG
TTC
SEQ ID NO: 6:
AGT ACA CAC AAG CAG TTC GAG TCC ATC
CTC AAA ATC CAG AAG ACA GGG AAA GGC
AAG AAG AAG GGG

Among the second base sequences, the base sequence having the sequence identity with the base sequence of SEQ ID NO: 5 is at the 5′ end side, and the base sequence having the sequence identity with the base sequence of SEQ ID NO: 6 is at the 3′ end side. Between these two base sequences, no base sequence may be inserted (that is, the latter base sequence may be present immediately after the former base sequence), but, for example, 15 to 75 bases (corresponding to 5 to 25 amino acid residues), particularly 21 to 60 bases (corresponding to 7 to 20 amino acid residues), more particularly 30 to 45 bases (corresponding to 10 to 15 amino acid residues), and more particularly 36 bases (corresponding to 12 amino acid residues) may be inserted.

The second base sequence encoding a region comprising both the transmembrane domain and the intracellular domain includes a base sequence having 70% or more, more preferably 80% or more, even more preferably 85% or more, particularly preferably 90% or more, 92% or more, 94% or more, 95% or more, 96% or more, 97% or more, 98% or more, or 99% or more sequence identity with the base sequence of SEQ ID NO: 4. Amino acid sequences encoded by such base sequences are preferred for binding to non-human animal FcεRI γ-chains.

SEQ ID NO: 4 is as follows. SEQ ID NO: 4 is a base sequence corresponding to amino acid residue numbers 206 to 246 (transmembrane domain and intracellular domain) in the amino acid sequence of SEQ ID NO: 8 (chimeric FcεRI α-chain protein of the present invention) described below.

SEQ ID NO: 4:
CTG ATC TTC CCT TCT
CTC GCA GTG ATT
CTG TTT GCC GTC GAC
ACC GGA CTG TGG
TTC AGT ACA CAC AAG
CAG TTC GAG TCC
ATC CTC AAA ATC CAG
AAG ACA GGG AAA
GGC AAG AAG AAG GGG

### (3) Third Base Sequence

The third base sequence encodes a signal sequence for allowing the chimeric FcεRI α-chain produced based on the gene to express on the cell membrane, as described above. For example, it may encode a signal sequence to effect cell membrane penetration (particularly single transmembrane penetration) of the chimeric FcεRI α-chain protein.

In one embodiment of the present invention, the signal sequence may be, for example, a signal sequence contained in a human FcεRI α-chain protein or a signal sequence contained in a non-human animal FcεRI α-chain protein. Preferably, the signal sequence is a signal sequence contained in the human FcεRI α-chain protein. The chimeric FcεRI α-chain protein produced from the chimeric FcεRI α-chain gene of the present invention is expressed on the cell membrane by the signal sequence encoded by the third base sequence.

In another embodiment of the present invention, the signal sequence may be a signal sequence contained in Igκ, e.g., a signal sequence contained in human Igκ or non-human animal Igκ. The signal sequence also allows the chimeric FcεRI α-chain protein to be expressed in the cell membrane.

The third base sequence preferably comprises a base sequence having 70% or more, more preferably 80% or more, even more preferably 85% or more, particularly preferably 90% or more, 92% or more, 94% or more, 95% or more, 96% or more, 97% or more, 98% or more, or 99% or more sequence identity with the base sequence of SEQ ID NO: 2. Such a base sequence is preferred for expression of the chimeric FcεRI α-chain protein to the cell membrane.

SEQ ID NO: 2 is as follows. SEQ ID NO: 2 is a base sequence corresponding to amino acid residue numbers 1 to 25 (signal sequence) in the amino acid sequence of SEQ ID NO: 8 (chimeric FcεRI α-chain protein of the present invention) described later.

SEQ ID NO: 2:
ATG GCA CCC GCA ATG GAG TCC CCC ACC
CTG CTG TGT GTC GCA CTG CTC TTT TTC
GCC CCC GAT GGA GTC CIC GCC

### (4) Entire Chimeric FcεRI α-Chain Gene

The chimeric FcεRI α-chain gene of the present invention preferably has a base sequence having 50% or more, more preferably 60% or more, even more preferably 70% or more, even more preferably 80% or more, still more preferably 85% or more, particularly preferably 90% or more, 92% or more, 94% or more, 95% or more, 96% or more, 97% or more, 98% or more, or 99% or more sequence identity with the base sequence of SEQ ID NO: 1.

SEQ ID NO: 1 is as follows. SEQ ID NO: 1 is a base sequence that encodes the amino acid sequence of SEQ ID NO: 8 (chimeric FcεRI α-chain protein of the present invention) described below.

```
SEQ ID NO: 1:
    1 ATG GCA CCC GCA ATG GAG TCC CCC ACC CTG CTG TGT GTC GCA CTG 45
  46 CTC TTT TTC GCC CCC GAT GGA GTC CTC GCC GTC CCC CAG AAG CCC 90
  91 AAG GTG TCT CTC AAC CCC CCT TGG AAT AGA ATC TTT AAA GGG GAG 135
136 AAC GTG ACC CTG ACA TGC AAT GGT AAC AAT TTC TTT GAA GTC AGC 180
181 TCC ACT AAG TGG TTT CAC AAC GGG TCA CTC AGC GAG GAA ACA AAT 225
226 TCT AGT CTG AAC ATT GTG AAT GCC AAG TTC GAG GAC AGC GGT GAA 270
271 TAC AAA TGC CAG CAT CAG CAG GTG AAC GAG TCC GAA CCT GTG TAT 315
316 CTG GAG GTC TTT TCC GAT TGG CTG CTC CTG CAG GCC TCT GCT GAG 360
361 GTG GTC ATG GAA GGC CAG CCA CTC TTC CTG AGG TGT CAC GGA TGG 405
406 CGG AAC TGG GAC GTG TAC AAG GTC ATC TAC TAT AAA GAT GGG GAG 450
451 GCC CTG AAG TAT TGG TAC GAA AAC CAT AAC ATC AGT ATC ACT AAC 495
496 GCA ACC GTG GAG GAC TCA GGC ACA TAC TAT TGT ACT GGA AAA GTC 540
541 TGG CAG CTC GAT TAC GAG AGC GAA CCA CTG AAT ATC ACC GTG ATT 585
586 AAG GCT CCC AGG GAG AAA TAT TGG CTC CAG CTG ATC TTC CCT TCT 630
631 CTC GCA GTG ATT CTG TTT GCC GTC GAC ACC GGA CTG TGG TTC AGT 675
676 ACA CAC AAG CAG TTC GAG TCC ATC CTC AAA ATC CAG AAG ACA GGG 720
721 AAA GGC AAG AAG AAG GGG TGA 741
```

SEQ ID NOs: 1-7 have been optimized taking into account rodent codon usage frequency, particularly rat codon usage frequency. Therefore, the non-human animal is preferably a rodent animal, more preferably a rat, mouse or hamster, particularly preferably a rat.

### (Production Method)

The chimeric FcεRI α-chain gene of the present invention can be produced, for example, by artificial gene synthesis techniques known in the art. For example, the chimeric FcεRI α-chain gene can be produced by chemical synthesis or enzymatic methods.

In addition, the chimeric FcεRI α-chain gene of the present invention may be produced by combining multiple genetic components using restriction enzymes and/or PCR, or may be produced by genome-editing the FcεRI α-chain gene of non-human animal cells (especially rat cells).

### 3. Second Embodiment (Chimeric FcεRI α-Chain Protein)

A configuration example of a chimeric FcεRI α-chain protein of the present invention is shown in FIG. 3B. The configuration example is as described in the above “2. First embodiment (chimeric FcεRI α-chain gene)”. That is, the chimeric FcεRI α-chain protein comprises the extracellular domain of the human FcεRI α-chain and the transmembrane domain of the non-human animal FcεRI α-chain. The chimeric FcεRI α-chain protein may further comprise an intracellular domain of a non-human animal FcεRI α-chain.

The extracellular domain may comprise the IgE binding region of the human FcεRI α-chain. In addition, the transmembrane domain can contribute to binding to the non-human animal FcεRI γ-chain of the non-human animal FcεRI α-chain.

Also, the chimeric FcεRI α-chain protein may further comprise a signal sequence for expression of the protein on the cell membrane.

### (1) Extracellular Domain

The extracellular domain comprises the IgE-binding region, which may be the region of the human FcεRI α-chain that binds to human IgE, as described above. The extracellular domain may correspond to, for example, the first base sequence contained in the chimeric FcεRI α-chain gene of the present invention described above. That is, the extracellular domain can have an amino acid sequence encoded by the first base sequence.

The extracellular domain may preferably comprise an amino acid sequence having 70% or more, more preferably 80% or more, even more preferably 85% or more, particularly preferably 90% or more, 92% or more, 94% or more, 95% or more, 96% or more, 97% or more, 98% or more, or 99% or more sequence identity with the amino acid sequence of SEQ ID NO: 14. Such an amino acid sequence functions as the IgE binding region.

SEQ ID NO: 14 is as follows. SEQ ID NO: 14 corresponds to amino acid residue numbers 110 to 183 in the amino acid sequence of SEQ ID NO: 8 (chimeric FcεRI α-chain protein of the present invention) described below. The amino acid sequence of SEQ ID NO: 14 is also the amino acid sequence encoded by the base sequence of SEQ ID NO: 7.

SEQ ID NO: 14:
Ser Asp Trp Leu Leu Leu Gln Ala Ser Ala Glu
Val Val Met Glu Gly Gln Pro Leu Phe Leu Arg
Cys His Gly Trp Arg Asn Trp Asp Val Tyr Lys
Val Ile Tyr Tyr Lys Asp Gly Glu Ala Leu Lys
Tyr Trp Tyr Glu Asn His Asn Ile Ser Ile Thr
Ash Ala Thr Val Glu Asp Ser Gly Thr Tyr Tyr
Cys Thr Gly Lys Val Trp Gln Leu

In a more preferred embodiment, the extracellular domain may be an amino acid sequence having 70% or more, more preferably 80% or more, even more preferably 85% or more, particularly preferably 90% or more, 92% or more, 94% or more, 95% or more, 96% or more, 97% or more, 98% or more, or 99% or more sequence identity with the amino acid sequence of SEQ ID NO: 10 below. The amino acid sequence is preferred for binding to IgE, and is also preferred for maintaining structure to provide IgE binding. The amino acid sequence may constitute the entire extracellular domain in the chimeric FcεRI α-chain protein of the present invention.

SEQ ID NO: 10 is the amino acid sequence of amino acid residue numbers 26 to 205 in the amino acid sequence of SEQ ID NO: 8 (chimeric FcεRI α-chain protein of the present invention) described below.

SEQ ID NO: 10:
Val Pro Gln Lys Pro Lys Val Ser Leu Asn Pro
Pro Trp Asn Arg Ile Phe Lys Gly Glu Asn Val
Thr Leu Thr Cys Asn Gly Asn Asn Phe Phe Glu
Val Ser Ser Thr Lys Trp Phe His Asn Gly Ser
Leu Ser Glu Glu Thr Asn Ser Ser Leu Asn Ile
Val Asn Ala Lys Phe Glu Asp Ser Gly Glu Tyr
Lys Cys Gln His Gln Gln Val Asn Glu Ser Glu
Pro Val Tyr Leu Glu Val Phe Ser Asp Trp Leu
Leu Leu Gln Ala Ser Ala Glu Val Val Met Glu
Gly Gln Pro Leu Phe Leu Arg Cys Ris Gly Trp
Arg Asn Trp Asp Val Tyr Lys Vai Ile Tyr Tyr
Lys Asp Gly Glu Ala Leu Lys Tyr Trp Tyr Glu
Asn His Asn Ile Ser Ile Thr Asn Ala The Val
Glu Asp Ser Gly Thr Tyr Tyr Cys Thr Gly Lys
Val Trp Gln Leu Asp Tyr Glu Ser Glu Pro Leu
Asn Ile Thr Val Ile Lys Ala Pro Arg Glu Lys
Tyr Trp Leu Gln

The extracellular domain may comprise two immunoglobulin domains. One of the two immunoglobulin domains can be formed by the amino acid sequence of amino acid residue numbers 44-108 in the amino acid sequence of SEQ ID NO: 8 (chimeric FcεRI α-chain protein of the present invention). The other of the two amino acid sequences can be formed by the amino acid sequence of amino acid residue numbers 121-194 in the amino acid sequence of SEQ ID NO: 8.

A region between these two amino acid sequences that functions as an immunoglobulin domain is also called a linker region. The linker region can be formed by the amino acid sequence of amino acid residue numbers 109-120 in the amino acid sequence of SEQ ID NO: 8.

These amino acid sequences also allow sequence identity as described above for SEQ ID NO: 10.

Such amino acid sequences are suitable for binding to IgE.

### (2) Transmembrane Domain and Intracellular Domain

The transmembrane domain may have a function of binding to the non-human animal FcεRI γ-chain. In order to perform the function, the transmembrane domain may comprise one or more amino acid residues that perform the function. For example, Asp contained in the transmembrane domain described below may be involved in the binding.

That is, the chimeric FcεRI α-chain protein of the present invention may comprise at least the transmembrane domain of the non-human animal FcεRI α-chain. The transmembrane domain may comprise one or more amino acid residues that function to bind to the non-human animal FcεRI γ-chain. The transmembrane domain may comprise, for example, the Asp as such amino acid residues.

Also, the chimeric FcεRI α-chain protein of the present invention may further comprise the intracellular domain of the non-human animal FcεRI α-chain. Particularly preferably, the chimeric FcεRI α-chain protein of the present invention comprises both the transmembrane domain and the intracellular domain of the non-human animal FcεRI α-chain.

The transmembrane domain may preferably comprise an amino acid sequence having 70% or more, more preferably 80% or more, even more preferably 85% or more, particularly preferably 90% or more, 92% or more, 94% or more, 95% or more, 96% or more, 97% or more, 98% or more, or 99% or more sequence identity with the amino acid sequence of SEQ ID NO: 12.

An amino acid sequence having the sequence identity with the amino acid sequence of SEQ ID NO: 12 corresponds to the transmembrane domain of the non-human animal FcεRI α-chain.

The intracellular domain may preferably comprise an amino acid sequence having 70% or more, more preferably 80% or more, even more preferably 85% or more, particularly preferably 90% or more, 92% or more, 94% or more, 95% or more, 96% or more, 97% or more, 98% or more, or 99% or more sequence identity with the amino acid sequence of SEQ ID NO: 13. Such an amino acid sequence is preferred for binding to non-human animal FcεRI γ-chains.

An amino acid sequence having the sequence identity with the amino acid sequence of SEQ ID NO: 13 corresponds to the intracellular domain of the non-human animal FcεRI α-chain.

SEQ ID NO: 12 and SEQ ID NO: 13 are as follows.

SEQ ID NO: 12 is amino acid residue numbers 206-224 (transmembrane domain) in the amino acid sequence of SEQ ID NO: 8 (chimeric FcεRI α-chain protein of the present invention) described below. The amino acid sequence of SEQ ID NO: 12 is also the amino acid sequence encoded by the base sequence of SEQ ID NO: 5.

SEQ ID NO: 13 is a base sequence corresponding to amino acid residue numbers 225 to 246 (intracellular domain) in the amino acid sequence. The amino acid sequence of SEQ ID NO: 13 is also the amino acid sequence encoded by the base sequence of SEQ ID NO: 6.

SEQ ID NO: 12:
Leu Ile Phe Pro Ser Leu Ala Val Ile
Leu Phe Ala Val Asp Thr Gly Leu Trp
Phe
SEQ ID NO: 13:
Ser Thr His Lys Gln Phe Glu Ser Ile
Leu Lys Ile Gln Lys Thr Gly Lys Gly
Lys Lys Lys Gly

From the viewpoint of a good binding property to non-human animal FcεRI γ-chain, the chimeric FcεRI α-chain protein of the present invention may comprise both the amino acid sequences of SEQ ID NO: 12 and SEQ ID NO: 13.

The amino acid sequence having the sequence identity with the amino acid sequence of SEQ ID NO: 12 may be on the N-terminal side, and the amino acid sequence having the sequence identity with the amino acid sequence of SEQ ID NO: 13 may be on the C-terminal side. Between these two amino acid sequences, no amino acid residue may be inserted (that is, the former amino acid sequence may be immediately followed by the latter amino acid sequence), but, for example, 5 to 25 amino acid residues, particularly 7 to 20 amino acid residues, more particularly 10 to 15 amino acid residues, more particularly 12 amino acid residues may be inserted.

When the chimeric FcεRI α-chain protein of the present invention comprises both the transmembrane domain and the intracellular domain of the non-human animal FcεRI α-chain, the protein may preferably comprise an amino acid having 70% or more, more preferably 80% or more, even more preferably 85% or more, particularly preferably 90% or more, 92% or more, 94% or more, 95% or more, 96% or more, 97% or more, 98% or more, or 99% or more sequence identity with the amino acid sequence of SEQ ID NO: 11.

Amino acid sequences having the sequence identity with the amino acid sequence of SEQ ID NO: 11 correspond to the transmembrane and intracellular domains of the non-human animal FcεRI α-chain.

SEQ ID NO: 11 is as follows.

SEQ ID NO: 11:
Leu Ile Phe Pro Ser Leu Ala Val Ile
Leu Phe Ala Val Asp Thr Gly Leu Trp
Phe Ser Thr Ris Lys Gln Phe Glu Ser
Ile Leu Lys Ile Glu Lys Thr Gly Lys
Gly Lys Lys Lys Gly

SEQ ID NO: 11 is amino acid residue numbers 206 to 246 (transmembrane domain and intracellular domain) in the amino acid sequence of SEQ ID NO: 8 (chimeric FcεRI α-chain protein of the present invention) described below. The amino acid sequence of SEQ ID NO: 11 is also the amino acid sequence encoded by the base sequence of SEQ ID NO: 4.

### (3) Signal Sequence

The signal sequence is, as described above, a signal sequence for expressing the chimeric FcεRI α-chain on the cell membrane. The signal sequence facilitates translocation of the chimeric FcεRI α-chain protein of the present invention to the cell membrane.

The signal sequence preferably comprises an amino acid sequence having 70% or more, more preferably 80% or more, even more preferably 85% or more, particularly preferably 90% or more, 92% or more, 94% or more, 95% or more, 96% or more, 97% or more, 98% or more, or 99% or more sequence identity with the amino acid sequence of SEQ ID NO: 9. Such an amino acid sequence is preferred for expression of the chimeric FcεRI α-chain protein to the cell membrane.

SEQ ID NO: 9 is as follows. SEQ ID NO: 9 is amino acid residue numbers 1 to 25 (signal sequence) in the amino acid sequence of SEQ ID NO: 8 (chimeric FcεRI α-chain protein of the present invention) described below. The amino acid sequence of SEQ ID NO: 9 is also the amino acid sequence encoded by the base sequence of SEQ ID NO: 2.

SEQ ID NO: 9:
Met Ala Pro Ala Met Glu Ser Pro Thr
Leu Leu Cys Val Ala Leu Leu Phe Phe
Ala Pro Asp Gly Val Leu Ala

### (4) Chimeric FcεRI α-Chain Protein

The chimeric FcεRI α-chain protein of the present invention preferably has an amino acid sequence with 70% or more, more preferably 80% or more, even more preferably 85% or more, particularly preferably 90% or more, 92% or more, 94% or more, 95% or more, 96% or more, 97% or more, 98% or more, or 99% or more sequence identity with the amino acid sequence of SEQ ID NO: 8.

SEQ ID NO: 8 is as follows.

```
SEQ ID NO: 8:
    1 Met Ala Pro Ala Met Glu Ser Pro Thr Leu Leu Cys Val Ala Leu 15
  16 Leu Phe Phe Ala Pro Asp Gly Val Leu Ala Val Pro Glo Lys Pro 30
  31 Lys Val Ser Leu Asn Pro Pro Trp Asn Arg Ile Phe Lys Gly Glu 45
  46 Asn Val Thr Leu Thr Cys Asn Gly Asn Asn Phe Phe Glu Val Ser 60
  61 Ser Thr Lys Trp Phe His Asn Gly Ser Leu Ser Glu Glu Thr Asn 75
  76 Ser Ser Leu Asn Ile Val Asn Ala Lys Phe Glu Asp Ser Gly Glu 90
  91 Tyr Lys Cys Gln His Gln Gln Val Asn Glu Ser Glu Pro Val Tyr 105
106 Leu Glu Val Phe Ser Asp Trp Leu Leu Leu Gln Ala Ser Ala Glu 120
121 Val Val Met Glu Gly Gln Pro Leu Phe Leu Arg Cys His Gly Trp 135
136 Arg Asn Trp Asp Val Tyr Lys Val Ile Tyr Tyr Lys Asp Gly Glu 150
151 Ala Leu Lys Tyr Trp Tyr Glu Asn His Asn Ile Ser Ile Thr Asn 165
166 Ala Thr Val Glu Asp Ser Gly Thr Tyr Tyr Cys Thr Gly Lys Val 180
181 Trp Gln Leu Asp Tyr Glu Ser Glu Pro Leu Asn Ile Thr Val Ile 195
196 Lys Ala Pro Arg Glu Lys Tyr Trp Leu Gln Leu Ile Phe Pro Ser 210
211 Leu Ala Val Ile Leu Phe Ala Val Asp Thr Gly Leu Trp Phe Ser 225
226 Thr His Lys Gln Phe Glu Ser Ile Leu Lys Ile Gln Lys Thr Gly 240
241 Lys Gly Lys Lys Lys Gly 246
```

Also, the chimeric FcεRI α-chain protein of the present invention may further comprise the intracellular domain of a non-human animal FcεRI γ-chain. As a result, the α-chain protein can play the role of the γ-chain in the cells in which the chimeric FcεRI α-chain protein of the present invention is expressed, and the γ-chain does not have to be expressed in the cells.

A chimeric FcεRI α-chain protein of the present invention may also further comprise the kinase domain of a tyrosine kinase such as Lyn or Syk. The kinase domain may be inserted downstream of the amino acid sequence having the sequence identity with the amino acid sequence of SEQ ID NO: 13. The kinase domain can regulate signaling in cells in which the chimeric FcεRI α-chain protein of the present invention is expressed, and can control allergic responses such as degranulation.

These components allow the induction of intracellular signaling independent of the host cell's gamma chain or tyrosine kinases. When these components are included, the chimeric FcεRI α-chain protein may preferably include, for example, a CD8 or IL2Ra transmembrane domain as a transmembrane domain for the induction.

A chimeric FcεRI α-chain protein of the present invention can be produced using a chimeric FcεRI α-chain gene of the present invention. For example, the chimeric FcεRI α-chain protein of the present invention can be produced by expressing the chimeric FcεRI α-chain gene of the present invention in a non-human animal cell from which the transmembrane domain encoded by the second base sequence contained in the gene is derived. For example, the chimeric FcεRI α-chain protein of the present invention can be expressed in the cell membrane of cells into which the gene has been introduced.

### 4. Third Embodiment (Vector)

The present invention also provides a vector comprising the chimeric FcεRI α-chain gene. The gene can be introduced into non-human animal cells by the vector. The vector may or may not be linearized upon introduction into non-human animal cells.

The vector may be produced, for example, by inserting the chimeric FcεRI α-chain gene into a plasmid known in the art. The plasmid has, for example, at least one cloning site, and the chimeric FcεRI α-chain gene can be inserted into the plasmid using the cloning site.

The type of plasmid may be appropriately selected by those skilled in the art according to the type of non-human animal cell, and examples of plasmids include pcDNA3.1(+) vector (Invitrogen).

A vector of the present invention may include, for example, a Kozak sequence. The Kozak sequence can increase the efficiency of translation initiation. The Kozak sequence may consist of, for example, the initiation codon of the chimeric FcεRI α-chain gene, 1 to 6 bases immediately before (5′ end side) the initiation codon, and 1 to 6 bases (especially 1 base) immediately after the initiation codon (3′ end side).

### 5. Fourth Embodiment (Cell)

The present invention also provides a non-human animal cell comprising the chimeric FcεRI α-chain gene, the chimeric FcεRI α-chain protein, or the vector.

The chimeric FcεRI α-chain gene, for example, may be integrated into a chromosome of the non-human animal cells, or may be integrated into a vector introduced into the non-human animal cells. For stable expression, preferably the chimeric FcεRI α-chain gene is integrated into the chromosome of the non-human animal cells.

The cell of the present invention preferably further comprises the FcεRI γ-chain gene of the non-human animal cell. Furthermore, the cells of the present invention can form a complex comprising the chimeric FcεRI α-chain protein produced based on the chimeric FcεRI α-chain gene and the FcεRI γ-chain protein of the cell. The complex may comprise one such α-chain and two such γ-chains. That is, the non-human animal FcεRI γ-chain protein, which is the expression product of the γ-chain gene, can form a complex with the chimeric FcεRI α-chain protein. The complex functions as an Fc receptor for human IgE and forms a complex that binds human IgE with high affinity. Allergic reactions occur when a single antigen binds to a plurality (e.g., two) of the IgE and crosslinks a plurality (e.g., two) of the complexes. Thus, the non-human cells can further comprise the γ-chain gene, and the non-human cells are capable of causing an allergic reaction, which is useful for performing various allergy tests.

The cell of the present invention more preferably further comprises the FcεRI β-chain gene of the non-human animal cell. The β-chain of non-human animal FcεRI produced based on the β-chain gene can enhance signaling by the cross-linking.

The γ-chain gene and the β-chain gene may be endogenous to the non-human animal, for example, naturally occurring in the chromosome of the non-human animal cell. For example, sequence information for rat γ and β-chain genes is available from the NCBI Reference Sequence database. For example, rat γ-chain gene information (including sequence information) is available online. Information (including sequence information) on the rat β-chain gene is available online In addition, amino acid sequence information of proteins expressed from these genes can be obtained from the uniprot database.

The cell of the present invention can form a complex comprising the chimeric FcεRI α-chain produced based on the chimeric FcεRI α-chain gene, the non-human animal FcεRI β-chain of the cell, and the non-human animal FcεRI γ-chain of the cell. The complex may comprise one α-chain, one β-chain and two γ-chains. That is, the complex is a tetramer, and the cells of the present invention may preferably be configured to form the tetramer. The tetramer may enhance signaling involved in the allergic reaction. As a result, an allergy test using the cells can be performed with higher sensitivity.

The cell of the present invention more preferably further comprises a reporter gene whose expression is induced by cross-linking between the complexes. The reporter gene allows highly sensitive allergy testing using the cells.

The reporter gene is, as described above, a gene whose expression is induced by cross-linking between the complexes. Genes induced in this way may preferably be constructed such that their expression is induced by a transcription factor activated by the cross-linking. The transcription factor may be any one selected from the group consisting of NF-AT, NF-κB, AP-1, Elk-1, Egr-1, GATA-1 and GATA-2, preferably NF-AT. Activation of NF-AT is suitable for detecting allergens among test substances that may induce type I allergy in subjects.

For example, the reporter gene may be under control of an enhancer to which the activating transcription factor can bind. A promoter may be placed upstream of the reporter gene. For example, a cell of the present invention comprises the enhancer and a promoter and reporter gene under the control of the enhancer. The enhancer, the promoter, and the reporter gene may be introduced into the cell, for example, by a vector (e.g., plasmid) having a region comprising these three elements (hereinafter also referred to as “reporter gene region”). The reporter gene region may be introduced into the chromosome of the non-human animal cell or may be present in a vector in the non-human animal cell. Preferably, the reporter gene region has been introduced into the chromosome of the non-human animal cell. This enables stable expression of the reporter gene. The reporter gene region can be introduced into the chromosome by, for example, homologous recombination.

The sequence of the enhancer may be appropriately selected by those skilled in the art according to the type of the transcription factor, and may be any of the sequences shown in Table 1 below, for example. The left column of Table 1 below lists the enhancer sequences. Each enhancer sequence is enclosed in parentheses, and the number outside the parentheses is an example of the number of repeats of the enhancer sequence. In the present technology, the number of repeats of an enhancer sequence of an enhancer contained in a reporter gene is not limited to the numbers listed in Table 1, and may be any number of, for example, 1 to 15, 2 to 15, particularly 2 to 10, and more particularly 3 to 10. The right column of Table 1 lists the transcription factors that bind to each enhancer sequence.

TABLE 1
Enhancer sequences

| | Transcription |
|---|---|
| Enhancer sequence | factor |
| SEQ ID NO: 17 (TGACTAA)₇ | AP-1 |
| SEQ ID NO: 18 (TGACTAA)₆ | AP-1 |
| SEQ ID NO: 19 (GGGGTGGGGN)₃ | Egr-1 |
| SEQ ID NO: 20 | NF-AT |
| (GGAGGAAAAACTGTTTCA | |
| TACAGAAGGCGT)₄ | |
| SEQ ID NO: 21 | NF-κB |
| (TGGGGACTTTCCGC)₅ | |

The promoter may have a sequence to which a complex comprising cellular RNA polymerase binds, for example, a promoter with a TATA box, preferably a promoter with the sequence TATATAA.

The reporter gene may be, for example, any one of a gene encoding an enzyme, a gene encoding a fluorescent protein, and a gene encoding a surface antigen (not endogenous to the non-human animal).

The gene encoding the enzyme may be, for example, any one or more of luciferase, ⊕-galactosidase, alkaline phosphatase, chloramphenicol acetyltransferase, and horseradish peroxidase.

The gene encoding the fluorescent protein may be, for example, any one or more of green fluorescent protein (GFP), Sirius, EBFP, ECFP, EGFP, Venus, and DsRed.

The non-human animal cells of the present invention may have one or more reporter genes of the same or different types. Preferably, the non-human animal cells of the present invention may comprise 1, 2, 3, 4, or 5 reporter genes.

The cell of the present invention may be a non-human animal cell. The non-human animal may, for example, be a rodent animal, preferably a rat, a mouse or a hamster, more preferably a rat.

The non-human animal cell of the present invention is preferably a cell configured so that the chimeric FcεRI α-chain protein and the FcεRI γ-chain protein derived from the non-human animal cell form a complex, more preferably a cell configured so that the chimeric FcεRI α-chain protein and the FcεRI β-chain protein and FcεRI γ-chain protein derived from the non-human animal cell form a complex. Such cell includes, for example, a mast cell, a basophil, a dendritic cell, an eosinophil, a monocyte, a macrophage, and a Langerhans cell. That is, the cell of the present invention may be any one of these enumerated cells, more preferably a mast cell, a basophil or a dendritic cell, even more preferably a mast cell.

The non-human animal cell of the present invention is preferably a rat mast cell, basophil, or dendritic cell, or a mouse mast cell, basophil, or dendritic cell, more preferably a rat or mouse mast cell.

The non-human animal cell of the present invention may be obtained, for example, by introducing the chimeric FcεRI α-chain gene into a cell isolated from an animal body or an established cell line. Examples of the established cell line that can be used in the present invention include, but are not limited to, rat mast cell line RBL-2H3 cells (cell number: JCRB0023, available from National Institute of Biomedical Innovation, Health and Nutrition JCRB cell bank, or cell number: CRL-2256, available from ATCC (trademark)) and mouse mast cell line MC/9 cells (available from ATCC (trademark)).

Non-human animal cells of the present invention may be, for example, cells deposited under the accession number: NITE BP-03230. The cells were internationally deposited at Independent administrative agency National Institute of Technology and Evaluation Patent Microorganism Depositary Center (NPMD) with a deposit date of Jun. 17, 2020.

The non-human animal cells of the present invention may be used, for example, in analysis methods, such as those described in “7. Sixth embodiment (analysis method)” below.

Also, the non-human animal cells of the present invention are suitable for use as cells in, for example, the method described in Patent Literature 1 above. That is, the non-human animal cells of the present invention may be used in the EXILE method described above.

### 6. Fifth Embodiment (Analysis Kit)

The present invention provides an analysis kit comprising the cells of the present invention. The kit may be used, for example, for analysis or diagnosis relating to allergy. The kit may be a kit used for performing at least one test selected from the group consisting of, for example, evaluation of allergenicity, evaluation of the presence or absence or risk of allergy, analysis of IgE, evaluation of allergy suppressive activity, and drug screening.

The kit of the present invention may comprise materials for detecting expression of a reporter gene. The detection material may comprise a substrate for the enzyme, e.g., if the reporter gene expresses an enzyme, e.g., a substrate for luciferase.

Kits of the present invention may include, for example, incubation materials used to incubate the cells of the present invention. The incubation material may refer to a material used to incubate the cells, e.g., the medium or buffer in which the cells are incubated. The incubation material may be, for example, an incubation material for sensitization incubation, an incubation material for cell response induction incubation. Also, the incubation material may be both a sensitizing and a cell response inducing incubation material.

Examples of the medium include, but are not limited to, MEM medium. The medium may comprise supplementary components such as, for example, fetal bovine serum.

Examples of the buffer include, but are not limited to, PBS. The PBS can be used, for example, as a cell wash.

Kits of the present invention may include a cell-sensitizing component. The component may be included in the incubation material for the sensitizing incubation mentioned above, e.g., in the medium or buffer. Alternatively, the components may be included in the kit without being included in media or buffers.

The cell-sensitizing component may be a component capable of sensitizing the cells of the present invention. The cell-sensitizing component is, for example, IgE or chimeric IgE. The IgE may be primate IgE, for example, human or non-human primate (especially monkey) IgE, preferably human IgE. The IgE may be purified or isolated IgE or may be IgE that is not purified or isolated. Also, the chimeric IgE may be, for example, a chimeric IgE in which the Fc portion is humanized.

The cell-sensitizing component may be a biomaterial, such as whole blood, serum, plasma, or a component contained in blood (e.g., blood cells).

A kit of the present invention may comprise a cell response-inducing component. The component may be contained in the incubation material for the cell response induction incubation described above, for example, in the medium or buffer. Alternatively, the components may be included in the kit without being included in media or buffers.

The cellular response-inducing component may be, for example, a component that binds to the cell-sensitizing component and induces a response of the cells of the present invention, such as a component that binds to the IgE or chimeric IgE. The cellular response-inducing component may be, for example, an anti-IgE antibody, or may be an antigen or antigen candidate that binds (e.g., specifically binds) to IgE or chimeric IgE.

The cellular response-inducing component, the antigen or antigen candidate substance may be, for example, a substance capable of causing an allergic reaction (especially a type I allergic reaction) in a subject (that is, a substance that may become an allergen). Such substances include, for example, grit; dust; skin waste such as dander; pollens such as cedar pollen, Alnus firma pollen, gramineous pollen, and Asteraceae pollen; fungi such as mold; insects such as chironomid and cockroach; irritating or toxic substances found in insects and seafood; substances contained in soybeans, eggs, wheat, milk, buckwheat, peanuts, shrimp, crab, etc.; substances contained in drugs such as penicillin; substances derived from animal bodies contained in excrement, etc.; vegetable substances such as flour and dust generated during wood processing; and other natural or chemically synthesized substances, but are not limited thereto. For example, when it is desired to detect whether or not a subject treats egg white as an allergen, purified products of substances present in egg white, such as ovalbumin, ovomucoid, and lysozyme, and egg white extracts can be used as test substances. When chimeric IgE is used as IgE, a low-molecular-weight hapten such as a nitrophenyl group can also be used as an antigen.

Kits of the present invention may further include positive and/or negative control materials for use in the test. A positive control material may comrise, for example, a calcium ionophore (such as ionomycin). A negative control material may be, for example, culture medium.

The kit of the present invention may comprise a substance that suppresses an activity of a transcription factor, for example, a substance that suppresses an activity of NF-AT. The substance may be, for example, cyclosporin A or tacrolimus. When the kit of the present invention is a kit for screening an antiallergic substance, the substance may be used as a positive control for the antiallergic substance.

Kits of the present invention may further comprise drugs.

The drug may be a sensitization inhibitor or a drug expected to inhibit sensitization. The drug may be, for example, a substance that binds to IgE or chimeric IgE, such as an anti-IgE antibody or an anti-chimeric IgE antibody.

The drug may be a cell response inhibitor or a drug expected to inhibit a cell response. The drug may be, for example, a signaling inhibitor that inhibits signaling caused by cross-linking of FcεRI.

The kit of the present invention may further comprise a container in which a test reaction is performed, particularly a container having a space in which the cells of the present invention are retained. Examples of such containers include well plates (96-well plates, etc.).

### 7. Sixth Embodiment (Analysis Method)

The present invention also provides analysis methods using cells according to the present invention. The method may be performed for analysis or diagnosis relating to allergy. The method may include, for example, carrying out, using the cells, evaluation of allergenicity, evaluation of the presence or absence or risk of allergy, analysis for IgE, evaluation of allergy suppressive activity, or drug screening. That is, the analysis method of the present invention may be configured as an allergenicity evaluation method, a method for evaluating presence or absence of allergy or risk of allergy, an IgE analysis method, a method for evaluating allergy suppressing activity, or a drug screening method.

The method of the present invention may, for example, be implemented to implement the method described in the above-mentioned Patent Literature 1, i.e., to implement the EXILE method.

Also, the method of the present invention may be performed for a second screening for antigen-specific IgE. For example, the methods of the present invention may be performed to evaluate whether the IgE is biologically significant or not, assay neutralizing antibodies, or assay cross-reactivity.

Also, the method of the present invention may be performed for drug screening, particularly high-throughput drug screening. For example, the method of the present invention may be performed for selecting or evaluating a substances that inhibits the binding of IgE and FcεRI, or for selecting or evaluating a substance that inhibits intracellular signaling of cells (especially mast cells, basophils, etc.).

The methods of the present invention may also be performed for basic scientific research. For example, the methods of the present invention may be performed for mechanism analysis or biomarker discovery.

A method of the present invention may comprise, for example, an incubation step of incubating cells according to the present invention and/or an analysis step of analyzing the response of the cells.

In the incubation step, for example, cells according to the present invention may be incubated. The incubation step may consist of two or more incubation steps, as described below.

The incubation step may comprise, for example, incubating the cells according to the present invention in an incubation material. As used herein, “incubation material” means a material used to incubate the cells, and may be, for example, a medium or buffer in which the cells are incubated.

In the analysis step, the response of cells according to the present invention may be analyzed. The analysis may be, for example, analysis of reporter gene expression as described above. The expression of the reporter gene may be the expression induced by the cross-linking between the complexes described in “5. Fourth embodiment (cell)” above. A concrete technique for analysis of reporter gene expression may be appropriately selected by those skilled in the art according to the type of the reporter gene. For example, when the reporter gene expresses an enzymatic protein, an analysis of the enzymatic reaction by the enzymatic protein can be performed in the analysis step. For example, when the enzymatic protein is luciferase, luminescence generated by substrate degradation by luciferase may be analyzed. For example, when the reporter gene expresses a fluorescent protein, the analysis step may involve analyzing the fluorescence generated by the fluorescent protein.

The analysis may also be an analysis of an allergic response in the cells, in particular an analysis of a type I allergic response. In the analysis, allergic responses such as degranulation may be analyzed.

The analysis step may be performed after the incubation step or may be performed while the incubation step is taking place. For example, the analysis step may be performed while any one of two or more incubation steps is being performed (particularly while the last incubation step is being performed).

In one embodiment of the present invention, the incubation step comprises two incubation steps. These two incubation steps are hereinafter also referred to as “first incubation step” and “second incubation step”. The first incubation step may be a sensitizing incubation step in which the cells according to the present invention are treated for sensitization. The second incubation step may be a cell response-inducing incubation step that induces a response in the treated cells.

In this embodiment, the analysis step may be performed, for example, while the first incubation step is being carried out, between the first incubation step and the second incubation step, while the second incubation step is being carried out, or after the second incubation step.

Each of these steps will be explained below.

### (1-1) First Incubation Step

The incubation material used in the first incubation step may be a material for sensitization or a material for analyzing sensitization. That is, the first incubation step includes incubating the cells in a material for sensitization or a material for analyzing sensitization. The material can be, for example, a medium or a buffer. The type of the medium or the buffer may be appropriately selected by those skilled in the art depending on, for example, the purpose of analysis. For example, the medium may be MEM medium. The material may comprise auxiliary components, such as fetal bovine serum (FBS). The material may comprise, for example, one or more antibiotics as auxiliary components.

As used herein, “material for sensitization” refers to a material used to sensitize the cells. The material for sensitization may mean a material comprising a component capable of sensitizing the cells (also referred to herein as a “cell-sensitizing component”).

The “cell-sensitizing component” is, for example, IgE or chimeric IgE. The IgE or chimeric IgE may be primate IgE or chimeric IgE, for example, human or non-human primate (especially monkey) IgE or chimeric IgE, preferably human IgE or chimeric IgE. The IgE or chimeric IgE may be purified or isolated IgE or chimeric IgE, or may be IgE or chimeric IgE that is not purified or isolated.

The “material containing a component capable of sensitizing cells” may be, for example, a medium or a buffer containing a cell-sensitizing component, particularly a medium containing the component.

In one embodiment of the present invention, the material for sensitization may be, for example, a material (particularly a medium or buffer) containing purified or isolated IgE.

In another embodiment of the present invention, the material for sensitization may be a material (medium or buffer) containing a biological component known to contain IgE (especially a primate-derived biological component, a human or non-human primate (monkey)-derived biological component), more specifically whole blood, serum, or plasma, or a material (medium or buffer) containing a blood component (e.g., blood cells).

In the present specification, the “material for analyzing sensitization” refers to a material that is the subject of analysis as to whether it can sensitize the cells. The material for analyzing sensitization may mean a material that may contain the cell-sensitizing component.

The “cell-sensitizing component” may be as described above.

The above-mentioned “material that may contain a cell-sensitizing component” may be a material (medium or buffer) for which it is unknown whether or not it contains the component, or a material that is known to contain the component but whose type or content is unknown. For example, the material may be a material found to contain or not contain the component, or a material for which the content and/or type of the component is found as a result of carrying out the method of the present invention.

### (1-2) Second Incubation Step

The incubation material used in the second incubation step may be a material for inducing cell response or a material for analyzing cell response induction. That is, the second incubation step includes incubating the cells in a material for inducing cell response or a material for analyzing cell response induction. The material can be, for example, a medium or a buffer. The type of the medium or the buffer may be appropriately selected by those skilled in the art depending on, for example, the purpose of analysis. For example, the medium may be MEM medium. The material may contain auxiliary components, such as fetal bovine serum (FBS). The material may contain, for example, one or more antibiotics as auxiliary components.

In the present specification, the “material for inducing cell response” may be a material containing a component that induces a cell response (hereinafter also referred to as a “cell response-inducing component”), particularly a component that induces a response of sensitized cells.

“The material for inducing cell response” may or may not contain the above-described cell-sensitizing component in addition to the cell response-inducing component.

The cellular response-inducing component may be, for example, a component that binds to the cell-sensitizing component and induces a response of the cells of the present invention, such as a component that binds to the IgE or chimeric IgE. The cellular response-inducing component may be, for example, an anti-IgE antibody, or may be an antigen or antigen candidate that binds (e.g., specifically binds) to IgE or chimeric IgE.

The cellular response induced by the cellular response-inducing component may be, for example, expression of the reporter gene described above. In particular, the expression may be an expression induced by cross-linking between the complexes.

The cellular response may also be an allergic response of the cells, in particular a cellular response relating to type I allergy. The cellular response may be an allergic response, e.g., degranulation.

In the present specification, the “material for analyzing cell response induction” refers to a material to be analyzed for its ability to induce a cell response. The “material for analyzing cell response induction” may mean, for example, a material that can contain a cell response induction component, and particularly a material that can contain a component that induces a response of sensitized cells.

The “cellular response-inducing component” may be as described above.

The “material that can contain a cell response-inducing component” may be a material (medium or buffer) for which it is unknown whether or not it contains the component, or a material that is known to contain the component but whose type and/or content is unknown. For example, the material may be a material found to contain or not contain the component, or a material for which the content and/or type of the component is found as a result of carrying out the method of the present invention.

### (1-3) Analysis Step

In the analysis step, a response of the cells is analyzed. The response may be, for example, expression of a reporter gene as described above. In particular, the expression may be an expression induced by cross-linking between the complexes. A specific technique for analysis of reporter gene expression may be appropriately selected by those skilled in the art according to the type of the reporter gene. For example, when the reporter gene expresses an enzymatic protein, an analysis of the enzymatic reaction may be performed in the analysis step. For example, when the enzymatic protein is luciferase, luminescence produced by substrate degradation by luciferase may be analyzed. For example, when the reporter gene expresses a fluorescent protein, fluorescence analysis may be performed in the analysis step.

The response may also be an allergic response of the cells, particularly a cellular response related to type I allergy, such as an increase in intracellular calcium ion concentration or degranulation. These allergic responses may be measured using fluorescent indicators.

Moreover, in order to analyze the response, an evanescent wave may be used to measure a change in the refractive index of a cell membrane associated with an allergic response.

### (1-4) Use of Drug

Drugs that may affect the response in the cells of the present invention may be used in the analysis methods of the present invention. The drug may be, for example, an agent used for inhibiting or suppressing allergic reactions, such as a sensitization inhibitor or a cell response inhibitor. The drug may be used, for example, in any one or more stages among
before the first incubation step,
during the first incubation step,
between the first and second incubation steps,
during the second incubation step, and
after the second incubation step.

By performing the method of the present invention using the drug, it is possible to evaluate the allergic reaction suppressing activity of the drug, for example. Drug screening can also be performed by carrying out the methods of the present invention using various drugs.

The agent can be used, for example, in the incubation material mentioned above. Examples of use of the drug in each step are as follows.

For example, in the first incubation step described in (1-1) above, the drug may be included in the sensitizing material or the sensitization analyzing material. In this case, the drug may be a sensitization inhibitor or a drug expected to inhibit sensitization. The drug can be, for example, a component that binds to IgE or chimeric IgE, such as an anti-IgE antibody or an anti-chimeric IgE antibody. Alternatively, the drug may be a drug that induces receptor downregulation. By using drugs in this way, it is possible to screen for drugs that have an action to suppress allergic reactions (particularly type I allergic reactions), or to evaluate the inhibitory action of allergic reaction inhibitors (particularly type I allergic reaction inhibitors).

Alternatively, the drug may be contacted with a cell-sensitizing component contained in the sensitizing material or the sensitization analyzing material prior to the first incubation step. The screening or the evaluation can also be carried out by incubating the cells in the sensitizing material or the sensitization analyzing material after the contacting.

Further, in the second incubation step described in (1-2) above, the drug may be contained in the cell response-inducing material or the cell response induction analysis material. In this case, the agent may be a cell response inhibitor or an agent expected to inhibit cell responses. The drug may be, for example, a signaling inhibitor that inhibits signaling by cross-linking of FcεRI. Examples of such drugs include, but are not limited to, calcineurin inhibitors such as, for example, cyclosporine and tacrolimus; tyrosine kinase inhibitors such as, for example, Genistein, Herbimycin A, and Piceatannol; PLC inhibitors such as, for example, U73122; PI3-kinase inhibitors such as, for example, Wortmannin; and, intracellular calcium ion chelators such as, for example, BAPTA-AM.

By using drugs in this way, it is also possible to screen drugs that have an action to suppress allergic reactions (particularly type I allergic reactions), or to evaluate the inhibitory action of allergic reaction inhibitors (particularly type I allergic reaction inhibitors).

Alternatively, between the first incubation step and the second incubation step, the drug may be brought into contact with the cell response-inducing material or the cell-response-inducing component contained in the cell response induction analysis material. The screening or the evaluation can also be performed by incubating the cells in the cell response-inducing material or the cell response induction analysis material after the contact.

### (1-5) Example of Allergenicity Evaluation

In this example, the cell response-inducing component (especially antigen or antigen candidate substance) used in the second incubation step is treated as a test sample, and the allergenicity of the cell response-inducing component can be evaluated.

In this example, in the first incubation step, cells according to the present invention are incubated in a material for sensitization containing, for example, IgE or IgE-containing serum obtained from a subject (human). This sensitizes the cells with the subject's IgE.

Next, in the second incubation step, the sensitized cells are incubated in a material for inducing cell response containing an antigen, which is a test sample. As a result, by the antigen, a complex is formed as described above in the cell membrane of the cell according to the present invention, and the formation of the complex allows expression of, for example, a reporter gene.

Then, cellular responses are analyzed in the analysis step. For example, in the analysis step, expression of the reporter gene may be analyzed. Analysis of the expression allows evaluation of the allergenicity of the antigen for the subject.

### (1-6) Evaluation of Presence or Absence or Risk of Allergy

In this example, the cell-sensitizing component (especially IgE or IgE-containing biological component) used in the first incubation step is treated as a test sample, and the presence or absence or risk of allergy (especially type I allergy) to a predetermined antigen in the subject from which the cell-sensitizing component is derived can be evaluated.

In this example, in the first incubation step, the cells according to the present invention are incubated in a sensitizing material containing IgE or an IgE-containing biological component (particularly IgE-containing serum), for example, obtained from a subject (human). This sensitizes the cells with the subject's IgE.

Next, in the second incubation step, the sensitized cells are incubated in a cell response-inducing material containing a predetermined antigen. As a result, the antigen forms a complex as described above in the cell membrane of the cell according to the present invention, and the formation of the complex allows expression of, for example, a reporter gene.

Next, in the analysis step, the expression of the reporter gene is analyzed. By analysis of the expression, the subject's presence or absence or risk of allergy to the predetermined antigen can be evaluated.

### (1-7) Analysis for IgE

In this example, the cell-sensitizing component (especially IgE or IgE-containing biological component) used in the first incubation step is treated as a test sample, and the cell-sensitizing component itself can be analyzed.

In this example, in the first incubation step, the cells according to the present invention are incubated in a sensitizing material containing IgE or an IgE-containing biological component (particularly IgE-containing serum), for example, obtained from a subject (human). This sensitizes the cells with the subject's IgE.

Next, in the second incubation step, the sensitized cells are incubated in a cell response-inducing material containing a predetermined antigen. As a result, the antigen forms a complex as described above in the cell membrane of the cell according to the present invention, and the formation of the complex allows expression of, for example, a reporter gene.

Next, in the analysis step, the expression of the reporter gene is analyzed. An evaluation of IgE can be made by analyzing the expression.

### (1-8) Evaluation Method for Allergy Suppressing Activity

In this example, a drug is treated as a test sample, and the allergic reaction suppressing activity of the drug can be evaluated.

In this example, in the first incubation step, the cells according to the present invention are incubated in a sensitizing material, e.g., containing IgE or an IgE-containing biological component (especially IgE-containing serum). This sensitizes the cells with the subject's IgE.

Next, in the second incubation step, the sensitized cells are incubated in a predetermined cell response-inducing material. As a result, the antigen forms a complex as described above in the cell membrane of the cell according to the present invention, and the formation of the complex allows expression of, for example, a reporter gene.

Here, a substance having an allergy-suppressing activity or a substance (drug) that is considered to have an allergy-suppressing activity is contained in the sensitizing material or the cell response-inducing material. When the substance is included in the sensitizing material, the substance can act as a sensitization inhibitor. When the substance is contained in the cell response-inducing material, the substance can act as a cell response inhibitor.

Next, in the analysis step, the expression of the reporter gene is analyzed. By analysis of the expression, the anti-allergic activity of the substance can be evaluated.

### (1-9) Drug Screening

In this example, various drugs are treated as test samples, and the allergic reaction suppressing activity of the various drugs is evaluated. Then, a drug having better allergic reaction suppressing activity can be selected.

In this example, in the first incubation step, the cells according to the present invention are incubated in a sensitizing material, e.g., containing IgE or an IgE-containing biological component (especially IgE-containing serum). This sensitizes the cells with the subject's IgE.

Next, in the second incubation step, the sensitized cells are incubated in a predetermined cell response-inducing material. As a result, the antigen forms a complex as described above in the cell membrane of the cell according to the present invention, and the formation of the complex allows expression of, for example, a reporter gene.

Here, various substances considered to have allergy-suppressing activity are contained in the sensitizing material or the cell response-inducing material. When the various substances are contained in the sensitizing material, the various substances are expected to act as sensitization inhibitors. When the various substances are contained in the cell response-inducing material, the various substances are expected to act as cell response inhibitors.

Next, in the analysis step, the expression of the reporter gene is analyzed when each of the various substances is used. A substance having better anti-allergic activity can be selected from among the various substances by analyzing the expression.

### 8. Examples

### (1) Synthesis of Chimeric FcεRI α-Chain Gene

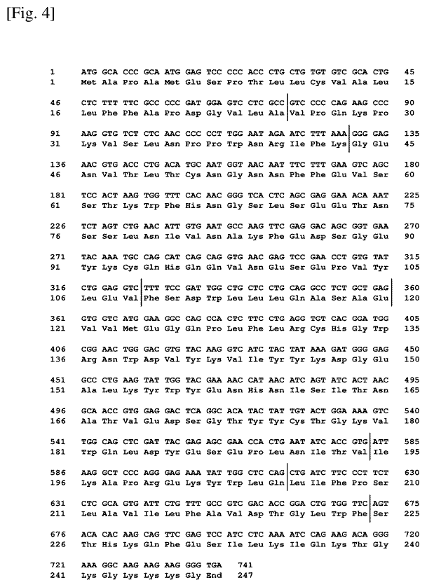

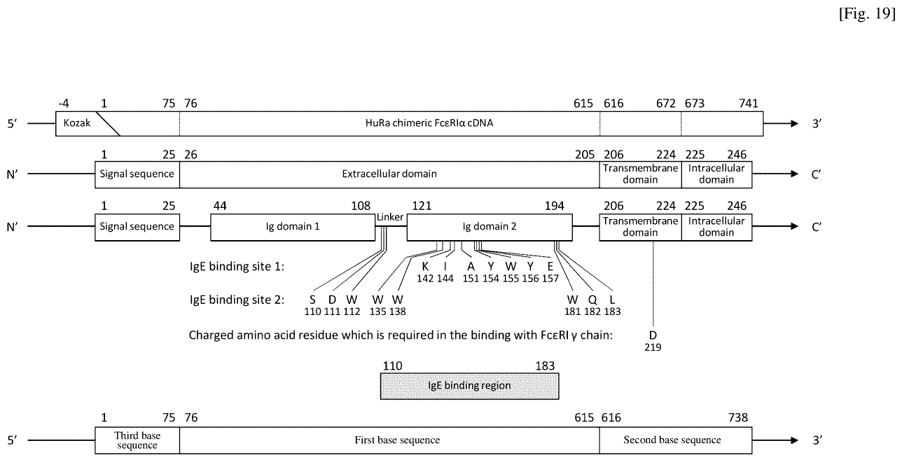

A chimeric receptor (Human-Rat chimeric FcεRI α-chain) gene was prepared by artificial gene synthesis, in which the extracellular domain of human FcεRI α-chain was linked to the transmembrane domain and intracellular domain of rat FcεRI α-chain. FIG. 4 shows the base sequence of the gene and the amino acid sequence encoded by the gene. In FIG. 4, the upper part shows the base sequence, and the lower part shows the amino acid sequence after translation. The base sequence and amino acid sequence shown in FIG. 4 are the same as the base sequence of SEQ ID NO: 1 and the amino acid sequence of SEQ ID NO: 8, respectively. FIG. 19 shows the relationship between the base sequence and amino acid sequence shown in FIG. 4 and the structure and function of the chimeric receptor protein.

As shown in FIG. 19, amino acid residue numbers 1 to 25 of the amino acid sequence shown in FIG. 4 are the signal sequence, amino acid residue numbers 26 to 205 are the extracellular domain, amino acid residue numbers 206 to 224 are the transmembrane domain, and amino acid residue numbers 225 to 246 are the intracellular domain.

The amino acid sequence of amino acid residue numbers 26-205 constituting the extracellular domain is the same as the sequence shown in SEQ ID NO: 10. The extracellular domain compirises an IgE binding region, and the IgE binding region corresponds to amino acid residue numbers 110-183. The amino acid sequence of amino acid residue numbers 110-183 is the same as the sequence shown in SEQ ID NO: 14. As shown in FIG. 19, among the amino acid residues contained in the IgE-binding region, particularly S110, D111, W112, W135, W138, K142, 1144, A151, Y154, W155, Y156, E157, W181, Q182, and L183 are amino acids involved in binding to IgE. Of these amino acids, K142, 1144, A151, Y154, W155, Y156, and E157 form a first IgE binding site, and S110, D111, W112, W135, W138, W181, Q182, and L183 form a second IgE binding site.

In addition, amino acid residue numbers 44-108 and 121-194 are immunoglobulin domains.

The amino acid sequence of the transmembrane domain is the amino acid sequence of the transmembrane domain contained in the rat FcεRI α-chain. The sequence of amino acid residue numbers 206-224 is the same as the sequence shown in SEQ ID NO: 12. An example of the base sequence encoding the amino acid sequence of the transmembrane domain is the sequence shown in SEQ ID NO: 5. As shown in FIG. 19, the transmembrane domain exerts binding properties with the γ-chain and the charged amino acid D219 is an amino acid specifically involved in a binding property to γ-chain.

The amino acid sequence of the intracellular domain is that of the intracellular domain contained in the rat FcεRI α-chain. The sequence of amino acid residue numbers 225-246 is the same as the sequence shown in SEQ ID NO: 13. An example of the base sequence encoding the amino acid sequence of the intracellular domain is the sequence shown in SEQ ID NO: 6.

The signal sequence is the signal sequence contained in the human FcεRI α-chain. The sequence of amino acid residue numbers 1-25 is the same as the sequence shown in SEQ ID NO: 9. Also, an example of the base sequence encoding the amino acid sequence of the signal sequence is the sequence shown in SEQ ID NO: 2.

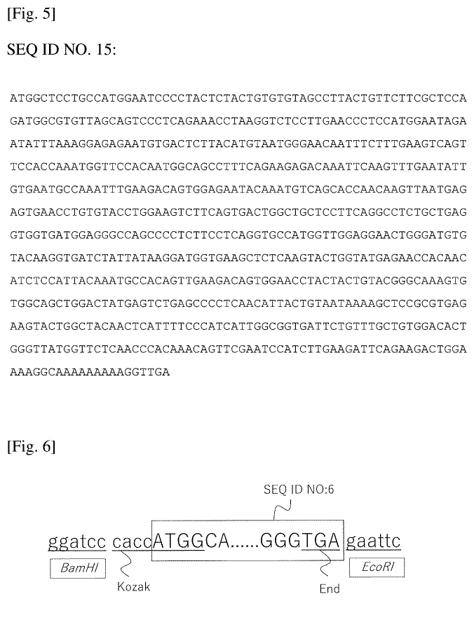

The base sequence of SEQ ID NO: 1 is optimized for efficient gene expression in cultured rat cells, taking rat codon usage frequency into consideration. Moreover, the base sequence before optimization is the base sequence of SEQ ID NO: 15 shown in FIG. 5. By comparing these two base sequences, the characteristics of the base sequence of SEQ ID NO: 1 will be described below.

The codon adaptation index (CAI) of the base sequence of SEQ ID NO: 15 is 0.72. On the other hand, the codon adaptation index of the base sequence of SEQ ID NO: 1 to rats is 0.83.

A higher codon adaptation index is preferred, with the maximum of 1, and preferably 0.8 or more, more preferably greater than 0.8, is considered good for high gene expression levels in cells.

The sequence of SEQ ID NO: 1 has a higher codon adaptation index than the sequence of SEQ ID NO: 15 and is therefore more suitable for expression in rat cells.

The codon adaptation index of the base sequence of the chimeric FcεRI α-chain gene of the present invention can be, for example, 0.7 or more, preferably 0.8 or more, more preferably 0.8 or more, still more preferably 0.81 or more, or 0.82 or more. The codon adaptation index may be, for example, 1.0 or less, 0.99 or less, or 0.95 or less.

The codon adaptation index can be used as a measure for estimating the relative expression level, based on the bias of the synonymous codon usage frequency of highly expressed genes such as ribosome-related genes, and from the correlation between the codon bias of these highly expressed genes and the codon bias of the genes to be observed. A codon adaptation index can be calculated, for example, according to the method described in P. M. Sharp and W. H. Li (Nucleic Acids Res. 1987 Feb. 11; 15(3): 1281-1295).

In the base sequence of SEQ ID NO: 15, the ratio of codons with a usage frequency (when the highest codon usage frequency is 100) of 91 to 100 is 47%. On the other hand, in the base sequence of SEQ ID NO: 1, the percentage of codons with usage frequencies of 91-100 is 59%.

A higher proportion of codons with high usage frequency is believed to be better for higher levels of gene expression in the cell.

The base sequence of SEQ ID NO: 1 has a higher percentage of codons with a usage frequency of 91-100 than the base sequence of SEQ ID NO: 15, and is thus more suitable for expression in rat cells.

In the chimeric FcεRI α-chain gene of the present invention, the percentage of codons with a usage frequency of 91 to 100 in cells in which the gene is expressed (particularly non-human animal cells) may be, for example, 40% or more, preferably 50% or more, more preferably 53% or more, and even more preferably 55% or more. The percentage can be, for example, 100% or less, 90% or less, 80% or less, 70% or less, or 60% or less.

The GC content of the base sequence of SEQ ID NO: 15 is 45.53%, whereas the GC content of the base sequence of SEQ ID NO: 1 is 51.05%. Higher GC content tends to result in longer half-life of transcript mRNA and is preferred for higher gene expression levels. Therefore, the base sequence of SEQ ID NO: 1 is considered to be able to achieve a higher gene expression level than SEQ ID NO: 15.

The GC content of the chimeric FcεRI α-chain gene of the present technology may be, for example, 40% or more, preferably 47% or more, more preferably 50% or more. The GC content can be, for example, 80% or less, 70% or less, or 60% or less.

The base sequence of SEQ ID NO: 15 comprises two sequences (AGTACT) recognized by restriction enzyme ScaI. On the other hand, the base sequence of SEQ ID NO: 1 does not comprise the above sequence.

The chimeric FcεRI α-chain genes of the present technology may comprise restriction enzyme sequences, but preferably do not comprise restriction enzyme sequences.

The base sequence of SEQ ID NO: 15 comprises three cis-acting elements (GGTGAT) for splicing, one poly-A signal cis-acting element (AATAAA), one cis-acting element for mRNA destabilization (ATTTA), and one poly-A signal cis-element (AAAAAAA). On the other hand, the base sequence of SEQ ID NO: 1 does not comprise these cis-elements. Therefore, in the base sequence of SEQ ID NO: 1, for example, a sequence that could be wrongly recognized as a splice site, a sequence that polyA could be erroneously added to, and a sequence that would lead to destabilization of mRNA were deleted.

Chimeric FcεRI α-chain genes of the present technology may comprise one or more of these cis-acting elements, but preferably do not comprise these cis-acting elements.

The base sequence of SEQ ID NO: 15 has a pair of inverted repeat sequences, but the base sequence of SEQ ID NO: 1 deos not have the sequence. Therefore, the latter can suppress the formation of unnecessary higher-order structures.

(2) Introduction of Chimeric FcεRI α-Chain Gene into RBL-NL4 Cells

As shown in FIG. 6, a nucleic acid (DNA) (SEQ ID NO: 16) was artificially synthesized having a base sequence in which a “recognition site for restriction enzyme BamHI (GGATCC)” and a “sequence for forming a KOZAK sequence (CACC)” were added to the 5′ end of SEQ ID NO: 1, and a “recognition site for restriction enzyme EcoRI (GAATTC)” was added to the 3′ end of SEQ ID NO: 1. The nucleic acid was inserted into a pcDNA3.1(+) vector (Invitrogen) having the two restriction enzyme recognition sites in the multiple cloning site, using the two sites. Vectors into which the nucleic acid was inserted were prepared in large quantities by techniques known in the art. The vectors into which the nucleic acid was inserted were then linearized using BamHI and EcoRI.

NF-AT (Nuclear factor activated T-cell)-dependent firefly luciferase expression vector (pHTS-NFAT, Biomyx) was stably introduced into RBL-2H3 cells (cell number: JCRB0023, JCRB cell bank, National Institute of Biomedical Innovation, Health and Nutrition) (hereinafter also referred to as “RBL-NL4 cells”). The linearized vector was introduced into the RBL-NL4 cells using Lipofectamine 3000, and drug selection was performed using hygromycin. Thereafter, cloning was performed by limiting dilution to obtain 7 clones into which the chimeric FcεRI α-chain gene was introduced.

RBL-NL4 cells express the endogenous rat FcεRI α, β, and γ-chain genes. Therefore, by expressing the introduced chimeric FcεRI α-chain gene, FcεRI in which the chimeric FcεRI α-chain protein produced by the expression of the gene associates with the β-chain and the γ-chain is functionally expressed on the cell membrane of the cell.

(3) Luciferase Expression in Cells into which Chimeric FcεRI α-Chain Gene was Introduced

Luciferase expression of when stimulated with ionomycin, anti-human IgE antibody, or specific antigen is confirmed for each of cultures obtained by culturing the seven clones obtained in (2) above and RBL-NL4 cells. Specifically, the confirmation was carried out as follows.

Each clone and RBL-NL4 cells were cultured in MEM medium supplemented with 10% inactivated fetal bovine serum. The composition of the medium is as follows.
MEM medium (Earle's salts (+), L-glutamine (−); Gibco 11090)
Fetal bovine serum (Gibco 10437-028, 30 minutes of inactivation at 56° C., working concentration 10%)
GlutaMAX-I (Gibco 35050)
Penicillin-streptomycin (Sigma P4333)
Geneticin (Gibco 10131, working concentration 500 μg/mL)
Hygromycin B (Hygromycin B, Invirtogen 10687-10, working concentration 200 μg/mL)
Penicillin and streptomycin were added to the medium to prevent bacterial infection. Geneticin and hygromycin B are selectable markers.

The culturing was performed in a plastic cell culture flask (T25) in an incubator at 37° C. in the presence of 5% carbon dioxide.

Cell passaging was performed as follows. That is, after decanting the culture supernatant (including floating cells) in the flask, 5 mL of fresh medium was added. After gently scraping the cells with a cell scraper and pipetting, 1/40 volume (0.125 mL) of the cells was added to a flask for passaging containing 5 mL of medium in advance, and cultured at 37° C. under 5% CO₂ for 5 days.

In addition, a T75 flask and double volume of medium (10 mL) were used for mass culture for use in various tests described later.

On the day before the test for confirming luciferase expression when the stimulus was applied, the following operations were performed. That is, purified human IgE (ab65866, Abcam) (50 ng/mL final concentration) was added to the MEM medium. The medium was placed in each well of a white 96-well plate made of plastic with a transparent bottom (product number: 165306, Thremo). Cells of each clone were then seeded at 5×10⁴ cells/50 μL per well, and allowed to stand overnight in an incubator for sensitization.

Also, in a similar way except that purified human IgE was not added, the cells of each clone were placed in each well of the 96-well plate and allowed to stand overnight in the incubator (i.e., non-sensitized cells were also prepared).

The next day, cells were washed multiple times with PBS. To the wells containing the sensitized cells, 100 ng/ml of anti-human IgE antibody prepared in the medium (product number A80-108A, BETHYL, final concentration 100 ng/mL) was added and the cells were stimulated at 37° C. for 3 hours. Similarly, 100 ng/ml of anti-human IgE antibody was also added to wells containing unsensitized cells and the cells were stimulated at 37° C. for 3 hours.

250 μM of ionomycin was also added to another well containing unsensitized cells and the cells were stimulated at 37° C. for 3 hours.

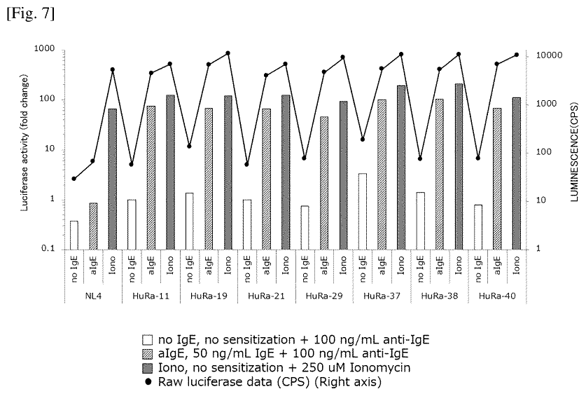

After applying the stimulation, 50 μL/well of a substrate solution (One-Glo Luciferase Assay System, Promega) was added to each well and allowed to react for 5 minutes, and then the luminescence intensity was measured using a luminometer (GloMax, Promega). The measurement results are shown in FIG. 7.

In FIG. 7, the measurement results for the seven clones are shown as “HuRa-11”, “HuRa-19”, “HuRa-21”, “HuRa-29”, “HuRa-37”, “HuRa-38”, and “HuRa-40”, respectively. The measurement results for RBL-NL4 cells are shown as “NL4”.

In FIG. 7, black dots indicate the measured luciferase luminescence intensity (unit: CPS) (right axis: LUMINESCENCE (CPS)). In FIG. 7, each bar indicates the relative luminescence intensity when anti-human IgE antibody was added to cells not sensitized with human IgE (no IgE), when sensitized cells were stimulated by adding anti-human IgE antibody (aIgE), and when cells not sensitized were stimulated with ionomycin (Iono) (left axis: Luciferase activity (fold change)). The relative luminescence intensity is the relative luminescence intensity when the luminescence intensity of each clone when unstimulated is set to 1, and is shown on a log scale.

As shown in FIG. 7, in comparison with sensitized (aIgE) and not sensitized (no IgE), anti-human IgE antibody stimulation increased luciferase expression levels by tens of times to 100 times for all clones. On the other hand, RBL-NL4 cells not expressing the chimeric FcεRI α-chain gene hardly responded to anti-human IgE antibody stimulation. Therefore, it can be seen that the introduction of the chimeric FcεRI α-chain gene imparted the ability to detect human IgE.

As shown in FIG. 7, the luciferase expression level in the case of stimulating RBL-NL4 cells with ionomycin (Iono) was greatly increased compared to the two cases in which RBL-NL4 cells were stimulated with anti-human IgE antibody (no IgE and aIgE). Ionomycin is a substance that activates NFAT without going through FcεRI. Therefore, it was confirmed that, in RBL-NL4 cells, the luciferase expression system functions normally due to the activation of NFAT.

In addition, among the above seven clones, HuRa-40 was particularly superior in terms of responsiveness to antibody stimulation, FcεRI expression level, and proliferation ability. HuRa-40 was deposited internationally at Independent administrative agency National Institute of Technology and Evaluation Patent Microorganisms Depositary Center (NPMD) under the accession number: NITE BP-03230 with a deposit date of Jun. 17, 2020.

(4) Confirmation of IgE-Binding Ability to Cells into which Chimeric FcεRI α-Chain Gene was Introduced

For each clone, binding of human IgE to the cell surface was measured by flow cytometry as follows.

First, for each clone, a cell sample that had been sensitized overnight with 50 ng/ml of human IgE and a cell sample that had not been sensitized were prepared. Each of these two cell samples was then contacted with 1 μg/mL of human IgE for 30 minutes on ice to allow all FcεRI to be occupied by IgE. Also, some of the non-sensitized cell samples were not exposed to human IgE on ice. Each sample was then stained with a FITC-labeled anti-human IgE antibody. Thus, the following three samples were prepared for each clone.
Sample 1: no sensitization, no human IgE contact on ice, with FITC staining
Sample 2: no sensitization, with human IgE contact on ice, with FITC staining
Sample 3: with sensitization, with human IgE contact on ice, with FITC staining

The above three samples were also prepared for RBL-NL4 cells (negative control) and RS-ATL8 cells (positive control).

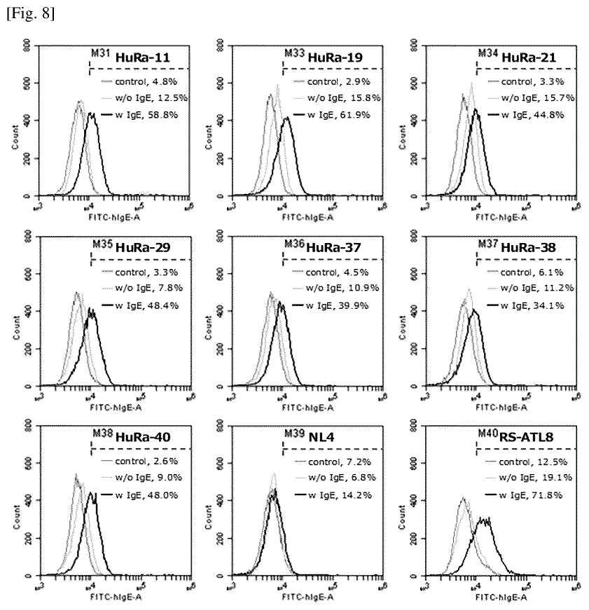

The fluorescence intensity of FITC was measured with a flow cytometer for the samples prepared as described above. The measurement results are shown in FIG. 8. As shown in FIG. 8, the number of cells in which FITC fluorescence was confirmed increased in the order of samples 1, 2, and 3, and in particular, the number of cells in which FITC fluorescence was confirmed for sample 3 was greatly increased compared to samples 1 and 2. Similar results were also obtained with RS-ATL8 cells as a positive control. On the other hand, such results were not obtained for the negative control RBL-NL4 cells. These results confirmed that IgE specifically bound to the cells of each clone.

### (5) Examination of Test Conditions Suitable for the Cells of the Present Invention

### (5-1) Stimulation Time

HuRa-40 cells suspended in 10% FBS-containing MEM medium containing 100-fold diluted healthy human serum (Cosmo Bio) were seeded in a 96-well microplate (ISOPLATE-96TC, PerkinElmer) at 5.0×10⁴ cells/50 μL/well and sensitized overnight in a CO₂ incubator.

After washing the sensitized cells, anti-human IgE antibody (0.1 μg/mL) diluted in MEM medium containing 10% FBS was added at 50 μL/well and stimulated for 1 hour, 3 hours, 6 hours, or 8 hours.

In addition, medium was added to unstimulated cells.

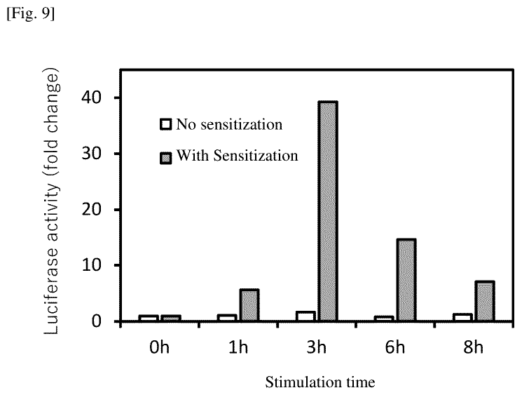

After the stimulation time had elapsed, a substrate solution of ONE-Glo EX Luciferase Assay System (Promega) was added to each well in an amount of 50 μL/well, and reacted for 5 minutes at room temperature while shielding from light. After that, the luminescence intensity was measured with a microplate reader (GloMax, Promega). The measurement results are shown in FIG. 9.

The relative luminescence intensity (Luciferase activity (fold change)) shown in FIG. 9 was calculated according to the following formula.

Luciferase activity (fold change)=(luminescence intensity of antibody-stimulated HuRa-40 cells at each time-luminescence intensity of medium)/(luminescence intensity of medium-added HuRa-40 cells at time 0-luminescence intensity of medium)

According to the results shown in FIG. 9, the stimulation time is preferably 1 hour or longer, more preferably 1.5 hours or longer, and even more preferably 2 hours or longer. The stimulation time may preferably be 8 hours or less, more preferably 7 hours or less, even more preferably 6 hours or less, 5 hours or less, or 4 hours or less. The stimulation time may be, for example, 1 hour to 8 hours, more preferably 2 hours to 8 hours, even more preferably 1.5 hours to 7 hours, 2 hours to 6 hours, 2 hours to 5 hours, or 2 hours to 4 hours, particularly preferably 3 hours to 4 hours.

### (5-2) Serum Concentration

HuRa-40 cells suspended in 10% FBS-containing MEM medium containing healthy human serum (Cosmo Bio) at various concentrations were seeded in a 96-well microplate (ISOPLATE-96TC, PerkinElmer) at 5.0×10⁴ cells/50 μL/well and sensitized overnight in a CO₂ incubator. Five serum concentrations were used: 0 vol %, 0.01 vol %, 0.1 vol %, 1 vol %, or 10 vol %.

After washing the sensitized cells, anti-human IgE antibody (0.1 μg/mL) diluted with 10% FBS-containing MEM medium was added at a volume of 50 μL/well and stimulated for 3 hours.

In addition, medium was added to unstimulated cells.

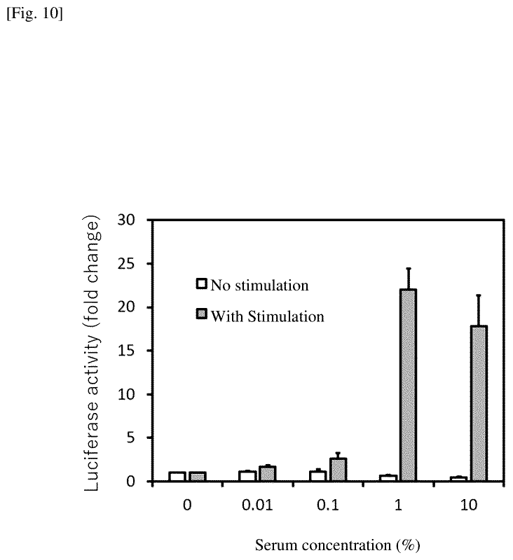

After the stimulation time had passed, the luminescence intensity was measured as described in (5-1) above. The measurement results are shown in FIG. 10. The relative luminescence intensity (Luciferase activity (fold change)) shown in FIG. 10 was calculated according to the following formula.

Luciferase activity (fold change)=(luminescence intensity of HuRa-40 cells at various serum concentrations-luminescence intensity of medium)/(luminescence intensity of HuRa-40 cells at serum concentration of 0 vol %-luminescence intensity of medium)

According to the results shown in FIG. 10, the serum concentration for sensitization is preferably 0.01 vol % or more, more preferably 0.1 vol % or more, even more preferably 0.5 vol % or more, 0.6 vol % or more, 0.7 vol % or more, 0.8 vol % or more, or 0.9 vol % or more. The serum concentration for sensitization may be, for example, 10 vol % or less, preferably 8 vol % or less, more preferably 7 vol % or less, even more preferably 6 vol % or less, 5 vol % or less, 4 vol % or less, 3 vol % or less, or 2 vol % or less. The serum concentration may be, for example, 0.1 vol % to 10 vol %, more preferably 0.5 vol % to 5 vol %, even more preferably 0.7 vol % to 3 vol %, particularly preferably 1 vol %.

### (5-3) Cytotoxicity

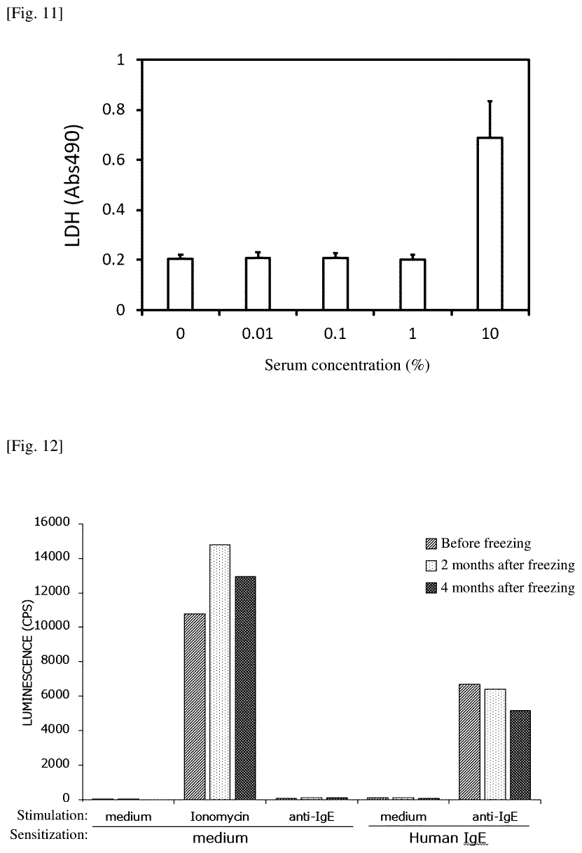

Cytotoxicity of human serum to HuRa-40 cells was evaluated using CytotoxicityLDH Assay Kit-WST (Dojindo Laboratories) and LDH released outside the cells as an index. The cell supernatant of the culture obtained by culturing the cells overnight in MEM medium containing human serum at each concentration (0 vol %, 0.01 vol %, 0.1 vol %, 1 vol %, or 10 vol %) was seeded in a 96-well microplate at 50 μL/well, Working Solution included in the kit was added to each well at 50 μL/well, and a color reaction was performed for 30 minutes at room temperature while shielding from light. Finally, 25 μL/well of Stop Solution included in the kit was added to each well, and absorbance at 490 nm was measured using a microplate reader (iMark, Bio Rad). The measurement results are shown in FIG. 11.

From FIG. 11, it can be seen that there is no toxicity at a serum concentration of 1% and that there is toxicity at a serum concentration of 10%.

### (5-4) Cryopreservation

The effect of −80° C. storage on the phenotypic stability of HuRa-40 cells was investigated. HuRa-40 cells were prepared before freezing at −80° C., 2 months after freezing, or 4 months after freezing. These three cells were sensitized overnight in MEM medium containing human IgE (ab65866, Abcam, final concentration 50 ng/mL). After the sensitization, the cells were washed with PBS three times. After washing, the cells were stimulated with an anti-human IgE antibody for 3 hours. Ionomycin (250 nM) was used as a positive control. After the stimulation, a substrate solution (One-Glo Luciferase Assay System, Promega) was added and the luminescence intensity was measured. The measurement results are shown in FIG. 12.

From the results shown in FIG. 12, the stability of the receptor-specific response during freezing for at least 4 months has been confirmed. It was also confirmed that receptor-specific responses of cells occurred even after storage at −80° C. for 8 months.

### (5-5) Passage Number

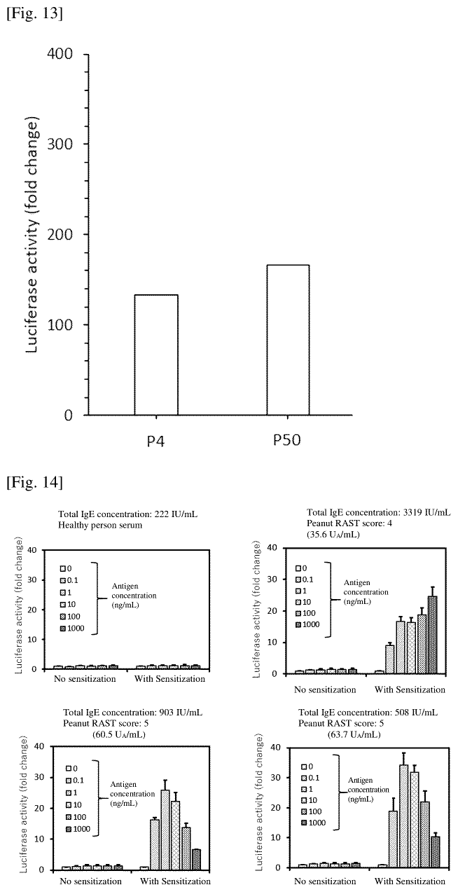

HuRa-40 cells at passage number 4 (P4) or passage number 50 (P50) were sensitized overnight by adding cedar pollinosis patient serum (specific IgE antibody titer of 100 or higher). After the sensitization, each cell was washed with PBS and stimulated with cedar pollen allergen extract. After the stimulation, a substrate solution (Bright-Glo, Promega) was added and luminescence was measured. The measurement results are shown in FIG. 13.

From the results shown in FIG. 13, no significant difference was observed between P4 and P50 in responses to the cedar pollen extract.

### (6) Test Using Allergy Patient Serum (Peanut Allergy)

(6-1) Antigen-Specific Response Using Patient Sera with Various RAST Scores

HuRa-40 cells suspended in MEM medium containing 10% FBS containing 100-fold diluted healthy human serum or peanut allergy patient serum with various RAST scores were seeded in a 96-well microplate (ISOPLATE-96TC, PerkinElmer) at 5.0×10⁴ cells/50 μL/well and sensitized overnight in a CO₂ incubator.

After washing the sensitized cells, allergen scratch extract (peanut) diluted with 10% FBS-containing MEM medium was added at various concentrations and stimulated at 37° C. for 3 hours.

The same stimulation treatment was also performed on cells that had not been sensitized.

After the stimulation time had elapsed, a substrate solution of ONE-Glo EX Luciferase Assay System (Promega) was added to each well in an amount of 50 μL/well, and reacted for 5 minutes at room temperature while shielding from light. After that, the luminescence intensity was measured with a microplate reader (GloMax, Promega). The measurement results are shown in FIG. 14.

From the results shown in FIG. 14, it can be seen that HuRa-40 cells respond in an antigen-specific manner.

### (6-2) Comparison Between the Cells of the Present Invention and Other Cells

Antigen-specific responses by HuRa-40 cells were compared to RS-ATL8 cells. In addition, RBL-NL4 cells were used as a negative control in the comparison.

RS-ATL8 cells are cells obtained by stably introducing an NF-AT-dependent firefly luciferase expression vector (Biomyx) into RBL-SX38 cells obtained by stably expressing human FcεRI in RBL-2H3 cells.

As described above, RBL-NL4 cells are host cells for HuRa-40 cells, and are RBL-2H3 cells obtained by stably introducing an NF-AT-dependent firefly luciferase expression vector (Biomyx).

Each of the three types of cells was suspended in 10% FBS-containing MEM medium containing peanut allergy patient serum (100-fold dilution) with a RAST score of 6 (>100 UA/mL), seeded in a 96-well microplate (ISOPLATE-96TC, PerkinElmer) at 5.0×10⁴ cells/50 μL/well, and sensitized overnight in a CO₂ incubator.

After washing the sensitized cells, an allergen scratch extract (peanut) diluted in MEM medium containing 10% FBS was added at various concentrations (0, 0.1, 1, 10, 100, or 1000 ng/mL) and stimulated at 37° C. for 3 hours.

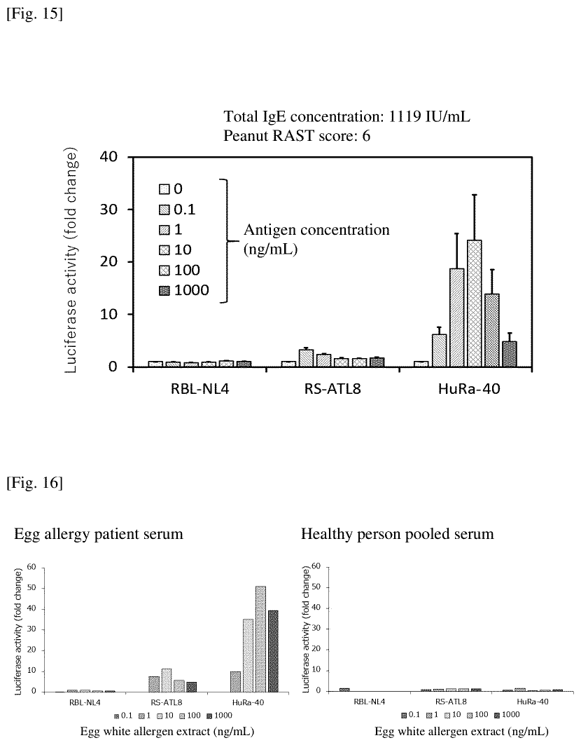

After the stimulation time had elapsed, a substrate solution of ONE-Glo EX Luciferase Assay System (Promega) was added to each well in an amount of 50 μL/well, and reacted for 5 minutes at room temperature while shielding from light. After that, the luminescence intensity was measured with a microplate reader (GloMax, Promega). The measurement results are shown in FIG. 15.

From the results shown in FIG. 15, it can be seen that HuRa-40 cells exhibit superior antigen responses compared to RS-ATL8 cells. Also, the responsiveness of HuRa-40 cells was superior to that of RS-ATL8 cells over a wide range of antigen concentrations.

### (7) Comparison Between the Cells of the Present Invention and Other Cells (Allergy Test, Egg Allergy)

Antigen-specific responses by HuRa-40 cells were compared to RS-ATL8 cells. In addition, RBL-NL4 cells were used as a negative control in the comparison.

Each of the three types of cells was suspended in 10% FBS-containing MEM medium containing egg-allergic patient serum (specific IgE antibody titer of 100 or more) or pooled serum from healthy persons, seeded in a 96-well microplate (ISOPLATE-96TC, PerkinElmer) at 5.0×10⁴ cells/50 μL/well, and sensitized overnight in a CO₂ incubator.

After washing the sensitized cells with PBS, egg white allergen extract diluted with 10% FBS-containing MEM medium was added at various concentrations (0.1, 1, 10, 100, or 1000 ng/mL) and stimulated at 37° C. for 3 hours.

After the stimulation time had elapsed, a substrate solution of ONE-Glo EX Luciferase Assay System (Promega) was added to each well in an amount of 50 μL/well, and reacted for 5 minutes at room temperature while shielding from light. After that, the luminescence intensity was measured with a microplate reader (GloMax, Promega). The measurement results are shown in FIG. 16.

From the results shown in FIG. 16, it can be seen that HuRa-40 cells show superior responsiveness compared to RS-ATL8 cells when using serum from egg-allergic patients. Moreover, no response to the egg white allergen extract was observed when pooled sera of healthy persons were used. These results show that HuRa-40 cells exhibit superior antigen-specific responsiveness than RS-ATL8 cells.

### (8) Comparison Between the Cells of the Present Invention and Other Cells (Evaluation of Effectiveness of Allergic Reaction Inhibitors)

Omalizumab is a treatment for bronchial asthma, seasonal allergic rhinitis, and chronic urticaria. Omalizumab is a humanized anti-human IgE monoclonal antibody that inhibits the binding of IgE to its high affinity receptor (FcεRI), thereby suppressing the activation of inflammatory cells such as basophils and mast cells. It competitively inhibits the binding of human IgE and FcεRI and reduces the concentration of free IgE in serum. It does not bind to IgE that has already bound to FcεRI. The cells of the present invention can be used, for example, to evaluate the effectiveness of allergic reaction inhibitors such as omalizumab. Experiments and their results showing that they can be used for the efficacy evaluation will be described below.

HuRa-40 cells and RS-ATL8 cells were prepared. Omalizumab at various final concentrations (0, 0.001, 0.01, 0.1, 1, 10, or 100 μg/mL) was added to each of these cells suspended in MEM medium containing 10% FBS, and 30 minutes after addition, 50 ng/ml of human IgE was added for overnight sensitization.

After washing the sensitized cells with PBS three times, a human IgE antibody was added and stimulated at 37° C. for 3 hours.

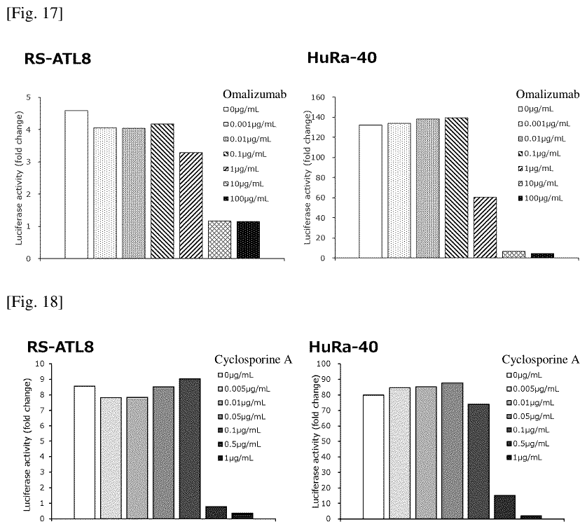

After the stimulation time had elapsed, Bright-Glo (Promega) substrate solution was added to each well in an amount of 50 μL/well, and reacted for 5 minutes at room temperature while shielding from light. After that, the luminescence intensity was measured with a microplate reader (GloMax, Promega). The measurement results are shown in FIG. 17.

According to the results shown in FIG. 17, luminescence intensity decreased slightly with increasing omalizumab concentration from 0.1 μg/mL to 1 μg/mL, and further decreased with increasing omalizumab concentration from 1 μg/mL to 10 μg/mL for RS-ATL8 cells. Furthermore, as can be seen from the figure, the 50% inhibitory concentration (IC50) of omalizumab is greater than 1 μg/mL for RS-ATL8 cells. On the other hand, as can be seen from the figure, for HuRa-40 cells, a significant decrease in luminescence intensity was already observed when the omalizumab concentration was increased from 0.1 μg/mL to 1 μg/mL. As can be seen from the figure, the IC50 of omalizumab is less than 1 μg/mL for HuRa-40 cells. Thus, HuRa-40 cells are able to detect the effects of omalizumab at lower concentrations compared to RS-ATL8 cells. Therefore, HuRa-40 cells are found to be more suitable for screening antiallergic substances than RS-ATL8 cells, and it is found that HuRa-40 cells can be used to screen antiallergic substances more sharply.

Further, according to the results shown in FIG. 17, with respect to RS-ATL8 cells, when the omalizumab concentration was the maximum (100 μg/mL), the luminescence intensity was only reduced to about ¼ compared to the case of 0 μg/mL. On the other hand, for HuRa-40 cells, the luminescence intensity at an omalizumab concentration of 100 μg/mL was significantly reduced to less than 1/30 of that at 0 μg/mL. That is, HuRa-40 cells leave little background compared to RS-ATL8 cells. These results also show that HuRa-40 cells are more suitable than RS-ATL8 cells for screening antiallergic active substances, and that HuRa-40 cells can be used to more sharply screen antiallergic active substances.

A study similar to that described above was performed with cyclosporin A instead of omalizumab. Specifically, RS-ATL8 cells or HuRa-40 cells were added with cyclosporin A at a final concentration of 0 to 1 μg/mL, and, 30 minutes later, 50 ng/ml of human IgE was added to sensitize them overnight. After washing with PBS three times, the cells were stimulated with an anti-human IgE antibody for 3 hours, and Bright-Glo was added to measure luminescence. The measurement results are shown in FIG. 18.

According to the results shown in FIG. 18, in HuRa-40 cells, addition of 0.1 μg/mL of cyclosporin A tended to suppress the response, and similar to RS-ATL8 cells, addition of 0.5 μg/mL of cyclosporin A significantly suppressed the response.

### (9) Stability and Reproducibility Precision

A phenotypic stability evaluation test and a reproducibility precision evaluation test in cryopreservation of HuRa-40 cells were performed as follows.

The luciferase assay used in these tests was performed as follows.

That is, an HuRa-40 cell suspension medium was added with 1/100 vol. of patient serum or Human IgE (ab65866, Abcam) (final concentration: 50 ng/mL), and seeded in a 96-well plate (165306, Thermo) so as to be 5.0×10⁴ cells/50 μL/well, and sensitized overnight at 37° C. in a CO₂ incubator. After the sensitization, the cells were washed three times with PBS, and after washing, 50 μL/well of egg white allergen extract or anti-human IgE antibody (A80-108A, BETHYL) prepared in medium (final concentration: 100 ng/mL) was added to each well and stimulated at 37° C. under CO₂ for 3 hours. After adding 50 μL/well of the following substrate solution and reacting for 5 minutes, the luminescence intensity was measured with a luminometer (GloMax, Promega).

### <Substrate Solution>

One-Glo Luciferase Assay System (Promega)
One-Glo EX Luciferase Assay System (Promega)
Bright-Glo (Promega)

### (Evaluation 1 of Stability of Phenotype of HuRa-40)

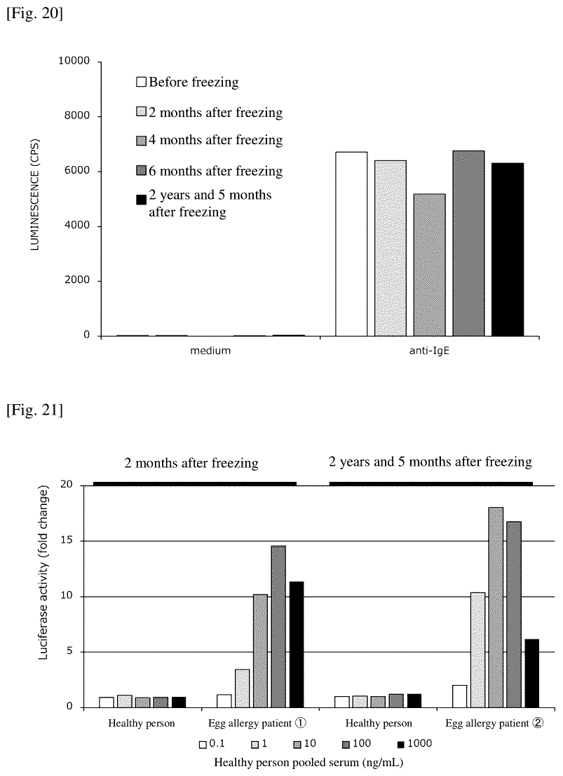

Human IgE was added to HuRa-40 cells before freezing, 2 months after freezing, 4 months after freezing, 6 months after freezing, or 2 years and 5 months after freezing to sensitize overnight. After washing with PBS three times, the cells were stimulated with an anti-human IgE antibody for 3 hours. One-Glo Luciferase Assay System was added to measure the luminescence intensity. The measurement results are shown in FIG. 20.

As shown in the figure, the stability of the receptor-specific response of HuRa-40 cells was confirmed for at least 2 years and 5 months.

### (Evaluation 2 of Stability of Phenotype of HuRa-40)

HuRa-40 cells 2 months after freezing or 2 years and 5 months after freezing were added with serum from egg-allergic patients (specific IgE antibody titer: 100 or higher) or pooled serum from healthy persons, and sensitized overnight, then, washed with PBS, stimulated with egg white allergen extract, and added with One-Glo EX Luciferase Assay System, to measure the luminescence intensity. The measurement results are shown in FIG. 21.

As shown in the figure, the stability of the receptor-specific response of HuRa-40 cells was confirmed for at least 2 years and 5 months.

### (Evaluation of Interlaboratory Reproducibility Precision)

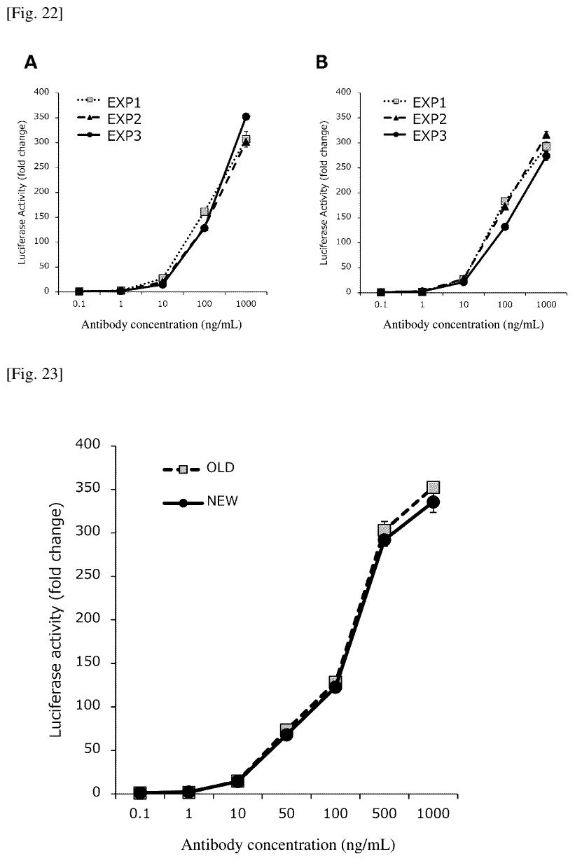

The following examination was conducted with 2 facilities (corresponding to A and B in FIG. 22) and 3 trial runs (corresponding to EXP 1, EXP 2 and EXP 3 in the same figure) (interlaboratory collaborative test). HuRa-40 cells were added with Human IgE, and sensitized overnight. After the sensitization, the cells were washed with PBS three times, and after the washing, they were stimulated with an anti-human IgE antibody for 3 hours. A homogeneous luciferase substrate solution (Bright-Glo) was added to measure the luminescence intensity. The measurement results are shown in FIG. 22.

As shown in the figure, the reproducibility precision of the test using HuRa-40 cells was very high.

### (Evaluation of Indoor Reproducibility Precision)

A single-laboratory reproducibility precision check was performed. HuRa-40 cells were sensitized overnight with different lots of human IgE (corresponding to OLD and NEW in FIG. 23). After the sensitization, the cells were washed with PBS three times, and after the washing, they were stimulated with an anti-human IgE antibody for 3 hours. A homogeneous luciferase substrate solution (Bright-Glo) was added to measure the luminescence intensity. The measurement results are shown in FIG. 23.

As shown in the figure, the reproducibility precision of the test using HuRa-40 cells was very high.

### [Accession Number]

Rat cells: NITE BP-03230

1. A chimeric FcεRI α-chain gene comprising:
a first base sequence encoding an extracellular domain of a human FcεRI α-chain and
a second base sequence encoding at least a transmembrane domain of a non-human.

2. The chimeric FcεRI α-chain gene according to claim 1, wherein the first base sequence comprises a base sequence having 70% or more sequence identity with the base sequence of SEQ ID NO: 7.

3. The chimeric FcεRI α-chain gene according to claim 1, wherein the second base sequence comprises at least a base sequence having 70% or more sequence identity SEQ ID NO: 5.

4. The chimeric FcεRI α-chain gene according claim 1, wherein the second base sequence further comprises a base sequence having 70% or more sequence identity with SEQ ID NO: 6.

5. The chimeric FcεRI α-chain gene according to claim 1, wherein the chimeric FcεRI α-chain gene further comprises a third base sequence encoding a signal sequence for expressing, on the cell membrane, the chimeric FcεRI α-chain generated based on the gene.

6. The chimeric FcεRI α-chain gene according to claim 5, wherein the third base sequence comprises a base sequence having 70% or more sequence identity with SEQ ID NO: 2.

7. The chimeric FcεRI α-chain gene according to claim 1, wherein the chimeric FcεRI α-chain gene has a base sequence having 50% or more sequence identity with SEQ ID NO: 1.

8. The chimeric FcεRI α-chain gene according to claim 1, wherein the non-human animal is a rodent animal.

9. A chimeric FcεRI α-chain protein comprising an extracellular domain of a human FcεRI α-chain and at least a transmembrane domain of a non-human animal FcεRI α-chain.

10. The chimeric FcεRI α-chain protein according to claim 9, wherein the extracellular domain comprises an amino acid sequence having 70% or more sequence identity with the amino acid sequence of SEQ ID NO: 14.

11. The chimeric FcεRI α-chain protein according to claim 9, wherein the transmembrane domain comprises an amino acid sequence having 70% or more sequence identity with the amino acid sequence of SEQ ID NO: 12.

12. The chimeric FcεRI α-chain protein according to claim 9, wherein the chimeric FcεRI α-chain protein further comprises an intracellular domain of a non-human animal FcεRI α-chain, and the intracellular domain comprises an amino acid sequence having 70% or more sequence identity with the amino acid sequence of SEQ ID NO: 13.

13. The chimeric FcεRI α-chain protein according to claim 9, wherein the chimeric FcεRI α-chain protein has an amino acid sequence having 70% or more sequence identity with the amino acid sequence of SEQ ID NO: 8.

14. A vector comprising the chimeric FcεRI α-chain gene according to claim 1.

15. A cell of a non-human animal comprising the chimeric FcεRI α-chain gene according to claim 1.

16. The cell according to claim 15, further comprising a gene of the γ-chain of FcεRI of the non-human animal cell.

17. The cell according to claim 16, which forms a complex comprising the chimeric FcεRI α-chain produced based on the chimeric FcεRI α-chain gene and the γ-chain of FcεRI of the cell.

18. (canceled)

19. An analysis kit comprising the cell according claim 15.

20. An analysis method using the cell according to claim 15, the method comprising performing an incubation step of incubating the cells and an analysis step of analyzing the response of the cells.

21. (canceled)

22. (canceled)

23. (canceled)

24. (canceled)


SEQUENCE LISTING (21 sequences)

SEQ ID NO: 1 - DNA, 741 bp, Artificial Sequence
  Chimeric FceRI a chain gene
atggcacccgcaatggagtcccccaccctgctgtgtgtcgcactgctctttttcgcccccgatggagtcctcgccgtcccccagaagcccaaggtgtctctcaaccccccttggaatagaatctttaaaggggagaacgtgaccctgacatgcaatggtaacaatttctttgaagtcagctccactaagtggtttcacaacgggtcactcagcgaggaaacaaattctagtctgaacattgtgaatgccaagttcgaggacagcggtgaatacaaatgccagcatcagcaggtgaacgagtccgaacctgtgtatctggaggtcttttccgattggctgctcctgcaggcctctgctgaggtggtcatggaaggccagccactcttcctgaggtgtcacggatggcggaactgggacgtgtacaaggtcatctactataaagatggggaggccctgaagtattggtacgaaaaccataacatcagtatcactaacgcaaccgtggaggactcaggcacatactattgtactggaaaagtctggcagctcgattacgagagcgaaccactgaatatcaccgtgattaaggctcccagggagaaatattggctccagctgatcttcccttctctcgcagtgattctgtttgccgtcgacaccggactgtggttcagtacacacaagcagttcgagtccatcctcaaaatccagaagacagggaaaggcaagaagaaggggtga

SEQ ID NO: 2 - DNA, 75 bp, Artificial Sequence
  Signal sequence of chimeric FceRI a chain gene
atggcacccgcaatggagtcccccaccctgctgtgtgtcgcactgctctttttcgcccccgatggagtcctcgcc

SEQ ID NO: 3 - DNA, 540 bp, Artificial Sequence
  Extracellular domain of chimeric FceRI a chain gene
gtcccccagaagcccaaggtgtctctcaaccccccttggaatagaatctttaaaggggagaacgtgaccctgacatgcaatggtaacaatttctttgaagtcagctccactaagtggtttcacaacgggtcactcagcgaggaaacaaattctagtctgaacattgtgaatgccaagttcgaggacagcggtgaatacaaatgccagcatcagcaggtgaacgagtccgaacctgtgtatctggaggtcttttccgattggctgctcctgcaggcctctgctgaggtggtcatggaaggccagccactcttcctgaggtgtcacggatggcggaactgggacgtgtacaaggtcatctactataaagatggggaggccctgaagtattggtacgaaaaccataacatcagtatcactaacgcaaccgtggaggactcaggcacatactattgtactggaaaagtctggcagctcgattacgagagcgaaccactgaatatcaccgtgattaaggctcccagggagaaatattggctccag

SEQ ID NO: 4 - DNA, 123 bp, Artificial Sequence
  Transmembrane and Intracellular domains of chimeric RceRI a chain gene
ctgatcttcccttctctcgcagtgattctgtttgccgtcgacaccggactgtggttcagtacacacaagcagttcgagtccatcctcaaaatccagaagacagggaaaggcaagaagaagggg

SEQ ID NO: 5 - DNA, 57 bp, Artificial Sequence
  Transmembrane domain of chimeric FceRI a chain gene
ctgatcttcccttctctcgcagtgattctgtttgccgtcgacaccggactgtggttc

SEQ ID NO: 6 - DNA, 66 bp, Artificial Sequence
  Intracellular domain of chimeric FceRI a chain gene
agtacacacaagcagttcgagtccatcctcaaaatccagaagacagggaaaggcaagaagaagggg

SEQ ID NO: 7 - DNA, 222 bp, Artificial Sequence
  IgE binding region of chimeric FceRI a chain gene
tccgattggctgctcctgcaggcctctgctgaggtggtcatggaaggccagccactcttcctgaggtgtcacggatggcggaactgggacgtgtacaaggtcatctactataaagatggggaggccctgaagtattggtacgaaaaccataacatcagtatcactaacgcaaccgtggaggactcaggcacatactattgtactggaaaagtctggcagctc

SEQ ID NO: 8 - protein, 246 aa, Artificial Sequence
  Chimeric FceRI a chain protein
MAPAMESPTLLCVALLFFAPDGVLAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQLIFPSLAVILFAVDTGLWFSTHKQFESILKIQKTGKGKKKG

SEQ ID NO: 9 - protein, 25 aa, Homo sapiens
MAPAMESPTLLCVALLFFAPDGVLA

SEQ ID NO: 10 - protein, 180 aa, Homo sapiens
VPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQ

SEQ ID NO: 11 - protein, 41 aa, Rattus norvegicus
LIFPSLAVILFAVDTGLWFSTHKQFESILKIQKTGKGKKKG

SEQ ID NO: 12 - protein, 19 aa, Rattus norvegicus
LIFPSLAVILFAVDTGLWF

SEQ ID NO: 13 - protein, 22 aa, Rattus norvegicus
STHKQFESILKIQKTGKGKKKG

SEQ ID NO: 14 - protein, 74 aa, Homo sapiens
SDWLLLQASAEVVMEGQPLFLRCHGWRNWDVYKVIYYKDGEALKYWYENHNISITNATVEDSGTYYCTGKVWQL

SEQ ID NO: 15 - DNA, 741 bp, Artificial Sequence
  preoptimized sequence of chimeric FceRI a chain gene
atggctcctgccatggaatcccctactctactgtgtgtagccttactgttcttcgctccagatggcgtgttagcagtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagctccgcgtgagaagtactggctacaactcattttcccatcattggcggtgattctgtttgctgtggacactgggttatggttctcaacccacaaacagttcgaatccatcttgaagattcagaagactggaaaaggcaaaaaaaaaggttga

SEQ ID NO: 16 - DNA, 757 bp, Artificial Sequence
  Seq_1 with 5′ BamHI and KOZAK sequences and 3′ EcoRI sequence
ggatcccaccatggcacccgcaatggagtcccccaccctgctgtgtgtcgcactgctctttttcgcccccgatggagtcctcgccgtcccccagaagcccaaggtgtctctcaaccccccttggaatagaatctttaaaggggagaacgtgaccctgacatgcaatggtaacaatttctttgaagtcagctccactaagtggtttcacaacgggtcactcagcgaggaaacaaattctagtctgaacattgtgaatgccaagttcgaggacagcggtgaatacaaatgccagcatcagcaggtgaacgagtccgaacctgtgtatctggaggtcttttccgattggctgctcctgcaggcctctgctgaggtggtcatggaaggccagccactcttcctgaggtgtcacggatggcggaactgggacgtgtacaaggtcatctactataaagatggggaggccctgaagtattggtacgaaaaccataacatcagtatcactaacgcaaccgtggaggactcaggcacatactattgtactggaaaagtctggcagctcgattacgagagcgaaccactgaatatcaccgtgattaaggctcccagggagaaatattggctccagctgatcttcccttctctcgcagtgattctgtttgccgtcgacaccggactgtggttcagtacacacaagcagttcgagtccatcctcaaaatccagaagacagggaaaggcaagaagaaggggtgagaattc

SEQ ID NO: 17 - DNA, 49 bp, Artificial Sequence
  Enhancer Sequence
tgactaatgactaatgactaatgactaatgactaatgactaatgactaa

SEQ ID NO: 18 - DNA, 42 bp, Artificial Sequence
  Enhancer Sequence
tgactaatgactaatgactaatgactaatgactaatgactaa

SEQ ID NO: 19 - DNA, 30 bp, Artificial Sequence
  Enhancer Sequence
  misc_feature (10)..(10): n is a, c, g, or t
  misc_feature (20)..(20): n is a, c, g, or t
  misc_feature (30)..(30): n is a, c, g, or t
ggggtggggnggggtggggnggggtggggn

SEQ ID NO: 20 - DNA, 120 bp, Artificial Sequence
  Enhancer Sequence
ggaggaaaaactgtttcatacagaaggcgtggaggaaaaactgtttcatacagaaggcgtggaggaaaaactgtttcatacagaaggcgtggaggaaaaactgtttcatacagaaggcgt

SEQ ID NO: 21 - DNA, 70 bp, Artificial Sequence
  Enhancer Sequence
tggggactttccgctggggactttccgctggggactttccgctggggactttccgctggggactttccgc